SEQUENCE LISTING (2 sequences)

SEQ ID NO: 1 - protein, 146 aa, Homo sapiens
MAGPLRAPLLLLAILAVALAVSPAAGSSPGKPPRLVGGPMDASVEEEGVRRALDFAVGEYNKASNDMYHSRALQVVRARKQIVAGVNYFLDVELGRTTCTKTQPNLDNCPFHDQPHLKRKAFCSFQIYAVPWQGTMTLSKSTCQDA

SEQ ID NO: 2 - protein, 120 aa, Homo sapiens
SSPGKPPRLVGGPMDASVEEEGVRRALDFAVGEYNKASNDMYHSRALQVVRARKQIVAGVNYFLDVELGRTTCTKTQPNLDNCPFHDQPHLKRKAFCSFQIYAVPWQGTMTLSKSTCQDA

(19) United States
(12) Patent Application Publication
(10) Pub. No.: US 2004/0171026 A1
(43) Pub. Date: Sep. 2, 2004

(54) Diagnostic method for transmissible spongiform encephalopathies

(76) Inventors: Denis Francois Hochstrasser, Geneva (CH); Jean-Charles Sanchez, Geneva (CH); Elisabeth Guillaume, Annemasse (FR)

Correspondence Address: BAKER & BOTTS, 30 ROCKEFELLER PLAZA, NEW YORK, NY, 10112

(21) Appl. No.: 10/695,194

(22) Filed: Oct. 28, 2003

Related U.S. Application Data
(63) Continuation-in-part of application No. PCT/EP02/10063, filed on Sep. 3, 2002.

(30) Foreign Application Priority Data
Sep. 5, 2001 (GB) 01 21459.2
Oct. 30, 2002 (GB) 02 25245.0
Mar. 19, 2003 (GB) 03 06290.8

(51) Int. Cl. C12Q 1/68

(52) U.S. Cl. 435/6

(57) ABSTRACT

Transmissible spongiform encephalopathy (TSE) is diagnosed in a subject by using mass spectrometry to observe a polypeptide in a sample of body fluid taken from the subject. The polypeptide is differentially contained in the body fluid of TSE-infected subjects and non-infected subjects, and has a molecular weight in the range of from 1000 to 100000.

### BACKGROUND OF THE INVENTION

This application is a continuation-in-part application of International Patent Application No. PCT/EP02/10063, filed Sep. 3, 2002 and published on Mar. 20, 2003 as WO03/023406, which claims priority from UK Patent Application No. 01 21459.2, filed Sep. 5, 2001.

### FIELD OF THE INVENTION

This invention relates to a method for obtaining information that may have utility in providing an indication of the presence of a transmissible spongiform encephalopathy (TSE) or the possibility or progress thereof.

### DESCRIPTION OF THE RELATED ART

Transmissible spongiform encephalopathies (TSEs) are neurodegenerative diseases of the central nervous system. They can be transmitted, inherited or occur sporadically and are observed in animals, e.g., as bovine spongiform encephalopathy (BSE) in cattle or scrapie in sheep, as well as in humans as Creutzfeldt-Jakob disease (CJD), Gerstman Sträussler Scheinker syndrome, Fatal Familial Insomnia or Kuru. They have a long incubation period, leading to ataxia, dementia, psychiatric disturbances and death. Neuropathological changes include vacuolar degeneration of brain tissue, astrogliosis and amyloid plaque formation. The diseases are difficult to diagnose pre-mortem.

The cerebrospinal fluid (CSF) of CJD patients displays two additional polypeptides (known as 14-3-3 polypeptides) by two-dimensional polyacrylamide gel electrophoresis [Harrington, M. G. New England Journal of Medicine 315, 279 (1986), Hsich, G., Kenney, K., Gibbs, C. J., Lee, K. H. & Harrington, M. B., New England Journal of Medicine 335, 924 (1996).] The function of these 14-3-3 polypeptides remains unclear in TSE. They can be used in a pre-mortem test for CJD diagnostic evaluation, but have low specificity.

Monoclonal antibodies to the abnormal form of prion protein (which is associated with CJD) are available and can be used in an enzyme-linked immunoassay, as described in PCT Specifications WO 98/23962 and 98/32710 and Schmerr, M. J., the Beckman Coulter Pace Setter Newsletter 3(2), 1-4 (June 1999), but these procedures have not yet been fully developed.

WO 01/67108 relates to a diagnostic assay for TSEs in which the concentration of heart or brain fatty acid binding protein (H-FABP or B-FABP) is determined in a sample of body fluid.

U.S. Pat. No. 6,225,047 describes the use of retentate chromatography to generate difference maps, and in particular a method of identifying analytes that are differentially present between two samples. One specific method described therein is laser desorption mass spectrometry.

WO 01/25791 describes a method for aiding a prostate cancer diagnosis, which comprises determining a test amount of a polypeptide marker, which is differentially present in samples of a prostate cancer patient and a subject who does not have prostate cancer. The marker may be determined using mass spectrometry, and preferably laser desorption mass spectrometry.

Development of new non-invasive TSE markers for body fluids or other body tissues (in particular, CJD and BSE markers in blood) and new methods of determining the markers would help clinicians to establish early diagnosis. This problem has now been solved by the present invention.

### SUMMARY OF THE INVENTION

The present invention provides a method of diagnosis of a transmissible spongiform encephalopathy (TSE) or the possibility thereof in a subject suspected of suffering from the TSE, which comprises subjecting a sample of body fluid taken from the subject to mass spectrometry, thereby to determine a test amount of a polypeptide in the sample, wherein the polypeptide is differentially contained in the body fluid of TSE-infected subjects and non-TSE-infected subjects, and has a molecular weight in the range of from 1000 to 100000; and determining whether the test amount is consistent with a diagnosis of TSE.

The invention also provides use of a polypeptide which is differentially contained in a body fluid of TSE-infected subjects and non-infected subjects, the polypeptide having a molecular weight in the range of from 1000 to 100000 and being determinable by mass spectrometry, for diagnostic, prognostic and therapeutic applications.

In embodiments of the invention, the molecular weight may, for example, be from 1000 to less than 3500, from 3500 to 30000, or from above 30000 to 100000.

The invention further relates to the use of a marker of molecular weight about 13350, reported in our application GB-A-2379737, in a method of diagnosis of a transmissible spongiform encephalopathy (TSE) or the possibility thereof in a subject suspected of suffering from TSE. That marker is believed to be cystatin C (Swiss-Prot Accession NO: P01034, active protein of 120 AA, theoretical pI of 8.75) also called Neuroendocrine basic polypeptide, Gamma Trace or Post gamma globulin. This secreted active inhibitor of cysteine proteinases belongs to a super-family of proteins that includes 3 groups on the basis of similar sequence and structural properties. This protein is highly expressed in the epididymis, vas deferens, brain, thymus, and ovary and at a lower level in the submandibular gland. Cystatin C has been confirmed by immunoblotting to be differentially expressed in the CSF of CJD affected patients. It is derived from a precursor having the sequence (SEQ ID No: 1):

| | |
|---|---|
| magplrapll llailavala vspaagsspg kpprlvggpm dasveeegvr raldfavgey | 60 |
| nkasndmyhs ralqvvrark qivagvnyfl dvelgrttct ktqpnldncp fhdqphlkrk | 120 |
| afcsfqiyav pwqgtmtlsk stcqda | 146 |

and has the sequence (SEQ ID No: 2) set out below:

| | |
|---|---|
| sspgkpprlv ggpmdasvee egvrraldfa vgeynkasnd myhsralqvv rarkqivagv | 60 |
| nyfldvelgr ttctktqpnl dncpfhdqph lkrkafcsfq iyavpwqgtm tlskstcqda | 120 |

A mutant form of cystatin C (Leu to Glu substitution in 68) has been described as being implicated in a hereditary form of cerebral hemorrhage characterized by a thickening of the cerebral artery walls, with deposition of material with the characteristics of amyloid. There are also some genotypes (BB: Ala to Thr substitution) associated with increased risk of late onset of Alzheimer disease.

The invention therefore provides a method of diagnosis of a transmissible spongiform encephalopathy (TSE) or the possibility thereof in a subject suspected of suffering from the TSE, which comprises subjecting a sample of body fluid taken from the subject to mass spectrometry, thereby to determine a test amount of a polypeptide in the sample, wherein the polypeptide is differentially contained in the body fluid of TSE-infected subjects and non-TSE-infected subjects, and is cystatin C; and determining whether the test amount is consistent with a diagnosis of TSE. The body fluid is preferably cerebrospinal fluid but may be whole blood, plasma, serum, urine or a tissue in which prion proteins tend to accumulate, e.g., the tonsil and other tissues of the lympho-reticular system such as the lymph nodes.

The invention further provides the use of a level of cystatin C measurable or detectable in a sample of body tissue by mass spectroscopy and differentially contained in the body tissue of TSE-infected subjects and non-TSE-infected subjects as a marker for providing an indication of a transmissible spongiform encephalopathy (TSE) or the possibility or progress thereof in a subject liable to suffer from the TSE.

In further experiments the inventors have identified and validated various hemoglobin isoforms as being the main protein able to discriminate between BSE+ and BSE−affected cattle using laser desorption/ionization mass spectrometry. In SELDI mass spectroscopy peaks or clusters at about 30,000, 15000 Da, 7500 Da may be indicative of hemoglobin, and these may correspond to a substantially intact hemoglobin molecule with a multiple electrical charge, or it may be a hemoglobin chain or a truncated version or fragment thereof having an immunological reaction to antibodies specific for bovine hemoglobin. The presence or absence of hemoglobin peaks or clusters in a SELDI spectrum therefore provides a means for ante-mortem diagnosis for BSE in cattle e.g., by tests carried out on plasma or other body fluids.

In a further aspect, therefore, the invention provides a method of diagnosis of a transmissible spongiform encephalopathy (TSE) or the possibility thereof in a bovine subject suspected of suffering from the TSE, which comprises subjecting a sample of body fluid taken from the subject to mass spectrometry, thereby to determine a test amount of a polypeptide in the sample, wherein the polypeptide is differentially contained in the body fluid of TSE-infected bovine subjects and non-TSE-infected subjects, and is a hemoglobin, a hemoglobin chain or a truncated chain or a fragment thereof thereof having an immunological reaction to antibodies specific for bovine hemoglobin; and determining whether the test amount is consistent with a diagnosis of TSE.

As used herein the expression “bovine” shall include cattle generally, sheep (scrapie) and also deer and elk (chronic wasting disease).

The invention further provides the use of a level of a hemoglobin, a hemoglobin chain or a truncated chain or a fragment thereof having an immunological reaction to antibodies specific for bovine hemoglobin, said level being measurable or detectable in a sample of body tissue by mass spectroscopy and said a hemoglobin, a hemoglobin chain or a truncated chain or a fragment thereof which exhibits an immunological reaction to an antibody to bovine hemoglobin being differentially contained in the body tissue of bovine TSE-infected subjects and non-bovine non-TSE-infected subjects as a marker for providing an indication of a transmissible spongiform encephalopathy (TSE) or the possibility or progress thereof in a bovine subject liable to suffer from the TSE.

The above test will be of value when applied to an animal or herd of animals either on a single occasion or at intervals, and animals that have been found not to be suffering from an actual or latent transmissible spongiform encephalopathy will self-evidently be of enhanced value. Such animals and the method of testing them are also within the scope of the invention.

The invention further provides assay devices or kits for use in the diagnosis of a TSE comprising a solid substrate having attached thereto an antibody that is specific for any of the following:

(i) a polypeptide that is differentially contained in the body fluid of TSE-infected subjects and non-TSE-infected subjects, and has a molecular weight in the range of from 1000 to 100000;

(ii) a polypeptide that is differentially contained in the body fluid of TSE-infected subjects and non-TSE-infected subjects, and is selected from those having a respective molecular weight of about 1010, 1100, 1125, 1365, 3645, 4030, 3890, 5820, 7520, 7630, 7980, 9950, 10250, 11600, 11800, 15000, 15200, 15400, 15600, 15900, 30000, 31000 and 31800 Da.;

(iii) cystatin C;

(iv) a hemoglobin, a hemoglobin chain or a truncated chain or a fragment thereof which exhibits an immunological reaction to an antibody to bovine hemoglobin and is differentially contained in the body tissue of bovine TSE-infected subjects and non-bovine non-TSE-infected subjects. The above devices or kits may further comprise necessary preparative reagents, washing reagents, detection reagents and signal producing reagents.

The invention also further provides an assay device for use in the diagnosis of TSE which comprises any of

a plate having a location containing a material which recognizes, binds to or has affinity for a polypeptide which is differentially contained in a body fluid of TSE-infected subjects and non-infected subjects, the polypeptide having a molecular weight in the range of from 1000 to 100000 and being determinable by mass spectrometry;

a plate having a location containing an antibody that is specific for Cystatin C;

a plate having a location containing an antibody that is specific for Cystatin C and useful in the diagnosis of variant CJD;

a plate having a location containing an antibody that is specific for Cystatin C and useful in the diagnosis of sporadic CJD.

The above devices may also be provided in association with necessary preparative reagents, washing reagents, detection reagents and signal producing reagents.

The invention further provides a kit for use in diagnosis of TSE, comprising a probe or a protein chip array for receiving a sample of body fluid, and for placement in a mass spectrometer, thereby to determine a test amount of a polypeptide in the sample, wherein the polypeptide is differentially contained in the body fluid of TSE-infected subjects and non-TSE-infected subjects, and has a molecular weight in the range of from 1000 to 100000.

### BRIEF DESCRIPTION OF THE DRAWINGS

FIG. 1 is a spectral view of CSF from normal and CJD-infected samples using laser desorption/ionization mass spectrometry;

FIG. 2 is a corresponding view highlighting a protein peak at about 4780 Da in CJD-infected CSF samples;

FIG. 3 is a corresponding view highlighting protein peaks at about 6700 and 8600 Da in CJD-infected CSF samples;

FIG. 4 is a corresponding view highlighting a protein peak at about 13375 Da in CJD-infected CSF samples;

FIG. 5 is a spectral view of plasma from normal and BSE-infected samples using laser desorption/ionization mass spectrometry;

FIG. 6 is a view corresponding to FIG. 5 and highlighting a protein peak at about 10220 Da in BSE-infected plasma samples;

FIG. 7 is a spectral view of plasma from CJD-infected patients (CJD+) and non-infected patients (CJD−) using laser desorption/ionization mass spectrometry;

FIGS. 8A and 8B are views corresponding to FIG. 7 and highlighting polypeptide peaks that are differentially expressed in the CJD+ and CJD− plasma samples;

FIGS. 9A to 9E are spectral views of plasma from CJD-infected patients (plasma CJD) and non-infected patients (plasma CTS) highlighting further polypeptide peaks that are differentially expressed in the infected and non-infected samples.

FIGS. 10A to 10F OF are spectral views of plasma from normal and BSE-infected samples using laser desorption/ionization mass spectrometry, highlighting protein peaks at about 1010, 1100, 1125, 1365, 3645, 4030, 3890, 5820, 7520, 7630, 7980, 9950, 10250, 11600, 11800, 15000, 15200, 15400, 15600, 15900, 30000, 31000 and 31800 Da in plasma samples;

FIG. 11 shows human cystatin C immunodetection in CSF samples;

FIG. 12 shows bovine hemoglobin detection in plasma samples;

FIG. 13 shows a BSE plasma sample on a two-dimensional gel and on a two-dimensional PVDF membrane;

FIG. 14 shows spectra of human and bovine hemoglobin using laser desorption/ionization mass spectrometry; and

FIGS. 15A to 15C show spectra from plasma from normal bovine and BSE-infected samples using laser desorption/ionization mass spectrometry.

### DESCRIPTION OF PREFERRED EMBODIMENTS

The invention provides a method of diagnosis of a transmissible spongiform encephalopathy (TSE) or the possibility thereof in a subject suspected of suffering from the TSE. A sample of body fluid taken from the subject is subjected to mass spectrometry, to determine the presence or absence in the sample of a polypeptide marker, which is differentially contained in the body fluid of TSE-infected subjects and non-infected subjects. The polypeptide marker has a molecular weight in the range of from 1000 to 100000, preferably from 1000 to 35000, and the presence, absence, under-expression or over-expression of the marker is indicative of TSE.

The method is applicable to all types of TSE, and to any human or animal suffering or suspected of suffering therefrom. The method is especially applicable to the diagnosis of CJD, especially new variant CJD, in human patients, and to BSE in ruminant animals such as cattle, and to BSE-like diseases in other animals, such as scrapie in sheep.

The term polypeptide includes proteins and protein fragments, as well as peptides modified by the addition of non-peptide residues, e.g., carbohydrates, phosphates, sulfates or any other post-translational modification.

The sample may be adsorbed on a probe under conditions which allow binding between the polypeptide and adsorbent material on the probe or the protein chip array. The adsorbent material preferably comprises a metal chelating group complexed with a metal ion, and a preferred metal is copper. Prior to detecting the polypeptide, unbound or weakly bound materials on the probe or protein chip array may be removed with a washing solution, thereby enriching the polypeptide in the sample. The sample is preferably adsorbed on a probe or protein chip array having an immobilized metal affinity capture (IMAC) surface capable of binding the polypeptide. The sample may be also adsorbed on a probe having hydrophobic, strong anionic or weak cationic exchange surfaces under conditions which allow binding of the polypeptides. The probe may consist of a strip having several adsorbent wells, and be inserted into the spectrometer, then movable therein so that each well is in turn struck by the ionizing means (e.g., laser) to give a spectrometer reading. The polypeptide is preferably determined by surface-enhanced laser desorption/ionization (SELDI) and time of flight mass spectrometry (TOF-MS).

In principle, any body fluid or tissue can be used to provide a sample for diagnosis, but preferably the body fluid is cerebrospinal fluid (CSF), plasma, serum, blood, urine, saliva or tears.

In one embodiment of the invention, the TSE is Creutzfeldt-Jakob disease (CJD). In this case, the polypeptide preferably has a molecular weight of about 4780, about 6700, about 8600 or about 13375, and the presence of one or more of such polypeptides is indicative of CJD. Alternatively, one or more polypeptides having a respective molecular weight of about 3970, about 3990, about 4294, about 4478, about 10075, about 11730, about 14043 or about 17839 is determined, and the absence of one or more of such polypeptides is indicative of CJD. As a further alternative, a polypeptide having a molecular weight of about 7770 is determined, and the presence of such polypeptide is indicative of CJD. According to one more example, CJD is indicated by a decrease in a peak at one or more of the following: about 3295, about 4315, about 4436, about 6200, about 8936, about 9107, about 9145, about 9185, about 9454 and about 13550 Da. According to yet a further example, CJD is indicated by an increase in a peak at one or more of the following: about 7574, about 7930, about 7975 and about 8020. It will be appreciated that the invention embraces making a measurement at any one or more of the foregoing molecular weight values, in any combination thereof, for the purpose of making a diagnosis of CJD.

In another embodiment of the invention, the TSE is bovine spongiform encephalopathy (BSE). In this case, the polypeptide preferably has a molecular weight of about 10220, and the presence of the polypeptide is indicative of BSE.

In a further embodiment of the invention, the TSE is bovine spongiform encephalopathy (BSE). In this case, the polypeptide preferably has a molecular weight of about 1010, 1100, 1125, 1365, 3645, 4030, 3890, 5820, 7520, 7630, 7980, 9950, 10250, 11600, 11800, 15000, 15200, 15400, 15600, 15900, 30000, 31000 and 31800 Da and the presence, absence, over-expression or under-expression of the polypeptide is indicative of BSE.

In a still further embodiment of the invention, the TSE is scrapie.

Measurement of the molecular weight of the polypeptide or polypeptides is effected in the mass spectrometer. All molecular weights herein are measured in Da. The molecular weights quoted above can be measured with an accuracy of better than 1%, generally 0.5 to 1%, and preferably to within about 0.1%. The term “about” in connection with molecular weights in this specification therefore means within a variation of about 1%, preferably 0.5%, and more preferably within about 0.1%, above or below the quoted value.

The invention also relates to the use of a polypeptide which is differentially contained in a body fluid of TSE-infected subjects and non-infected subjects, the polypeptide having a molecular weight in the range of from 1000 to 100000 and being determinable by mass spectrometry, for diagnostic, prognostic and therapeutic applications. This may involve the preparation and/or use of a material, which recognizes, binds to or has some affinity to the above-mentioned polypeptide. Examples of such materials are antibodies and antibody chips. The term “antibody” as used herein includes polyclonal antiserum, monoclonal antibodies, fragments of antibodies such as Fab, and genetically engineered antibodies. The antibodies may be chimeric or of a single species. The above reference to “prognostic” applications includes making a determination of the likely course of a TSE by, for example, measuring the amount of the above-mentioned polypeptide in a sample of body fluid. The above reference to “therapeutic” applications includes, for example, preparing materials which recognize, bind to or have affinity to the above-mentioned polypeptides, and using such materials in therapy. The materials may in this case be modified, for example by combining an antibody with a drug, thereby to target the drug to a specific region of the animal to be treated.

The methodology of this invention can be applied to the diagnosis of any TSE. Body fluid samples are prepared from infected and non-infected subjects. The samples are applied to a probe or array having a surface treated with a variety of adsorbent media, for differential retention of peptides in the sample, optionally using washing liquids to remove unbound or weakly bound materials. If appropriate, energy-absorbing material can also be applied. The probe or array is then inserted into a mass spectrometer, and readings are taken for the various sample/adsorbent combinations using a variety of spectrometer settings. Comparison of the infected and non-infected samples under a given set of conditions reveals one or more polypeptides, which are differentially expressed in the infected and non-infected samples. The presence or absence of these polypeptides can then be used in the testing of a fluid sample from a subject under the same conditions (adsorbent, spectrometer settings, etc.) to determine whether or not the subject is infected.

References herein to “presence or absence” of a polypeptide should be understood to mean simply that there is a significant difference in the amount of a polypeptide which is detected in the infected and non-infected sample. Thus, the “absence” of a polypeptide in a test sample may include the possibility that the polypeptide is actually present, but in a significantly lower amount than in a comparative test sample. According to the invention, a diagnosis can be made on the basis of the presence or absence of a polypeptide, and this includes the presence of a polypeptide in a significantly lower or significantly higher amount with reference to a comparative test sample.

The following Examples illustrate the invention.

### EXAMPLE 1

Polypeptides in Body Fluids (Cerebrospinal Fluid, Plasma and Others) of Creutzfeld-Jacob-Affected Patients

The objective of the present study was to detect specific polypeptides in body fluids (cerebrospinal fluid, plasma and others) of Creutzfeld-Jacob-affected patients. Samples were analyzed by the Surface Enhanced Laser Desorption Ionization (SELDI) Mass Spectroscopy (MS) technology. This technology encompasses micro-scale affinity capture of proteins by using different types of retentate chromatography and then analysis by time of flight mass spectrometry. Different maps are thus generated each corresponding to a typical protein profiling of given samples that were analyzed with a Ciphergen Biosystem PBS II mass spectrometer (Freemont, Calif., USA). Differential expressed peaks were identified when comparing spectra generated in a group of cerebrospinal fluid (CSF) samples from CJD-affected patients with a group of dementia-affected patients.

The SELDI analysis was performed using 21 μl of crude human CSF samples in order to detect specific polypeptides with metal affinity. An immobilized copper affinity array (IMAC-Cu⁺⁺) was employed in this approach to capture proteins with affinity for copper to select for a specific subset of proteins from the samples. Captured proteins were directly detected using the PBSII Protein Chip Array reader (Ciphergen Biosystems, Freemont, Calif., USA).

The following protocol was used for the processing and analysis of ProteinChip arrays using Chromatographic TED-Cu(II) adsorbent array. TED is a (tris(carboxymethyl) ethylenediamine-Cu) adsorbent coated on a silicon oxide-coated stainless steel substrate.

The surface was first loaded with 1011 of 100 mM copper sulfate to each spot and incubated for 15 minutes in a wet chamber.

The chip was thereafter washed by two quick rinses with deionized water for about 10 seconds to remove the excess unbound copper.

Before loading the samples, the I-MAC 3 array was equilibrated once with 5 μl of PBS NaCl 0.5 M for 5 minutes.

After removing the equilibration buffer, 3 μl of the same buffer were added before applying 2 μl of CSF. The chip was incubated for 20 minutes in a wet chamber.

The samples were thereafter removed and the surface was washed three times with the equilibration buffer (5 minutes each).

Two quick final rinses with water were performed.

The surface was allowed to air dry, followed by the addition of 0.5 μl of saturated sinapinic acid (SPA, Ciphergen Biosystem) prepared in 50% acetonitrile, 0.5% trifluoroacetic acid.

The chip was air dried again before analysis of the retained protein on each spot with laser desorption/ionization time-of-flight mass spectrometry.

The protein chip array was inserted into the instrument and analyzed once the appropriate detector sensitivity and laser energy have been established to automate the data collection.

The obtained spectra were analyzed with the Biomark Wizard software (Ciphergen Biosystems, Freemont, Calif., USA) running on a Dell Dimension 4100 PC. It generates consistent peak sets across multiple spectra.

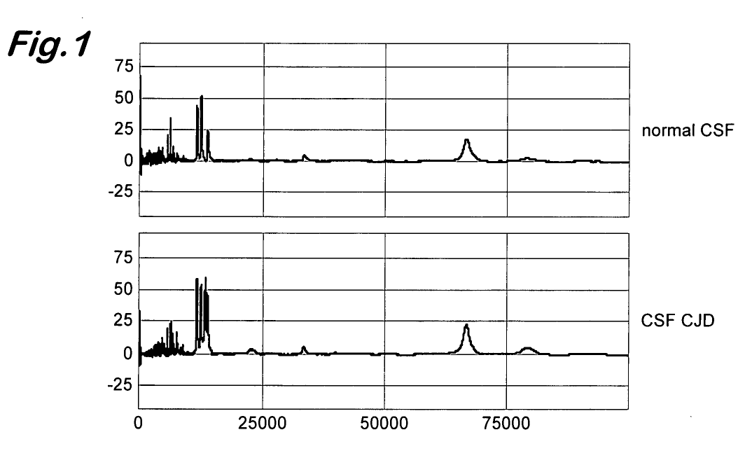

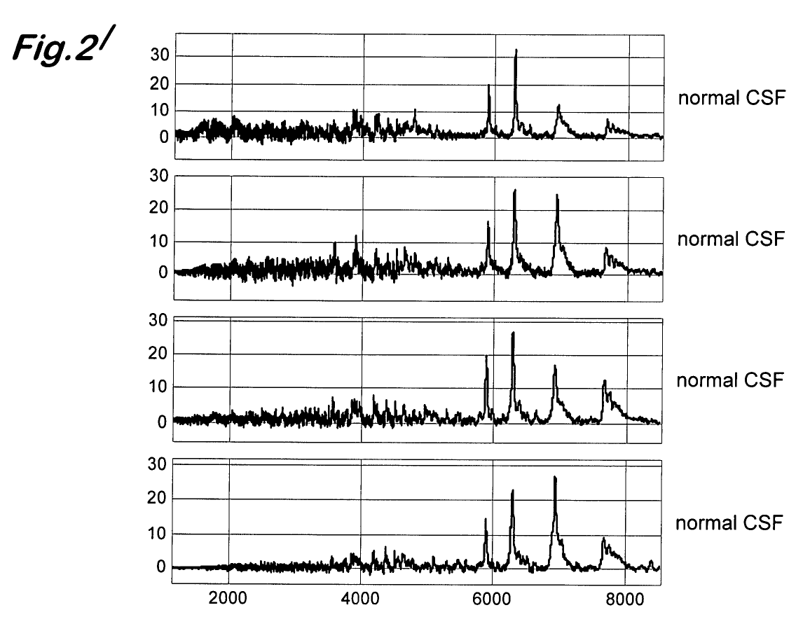

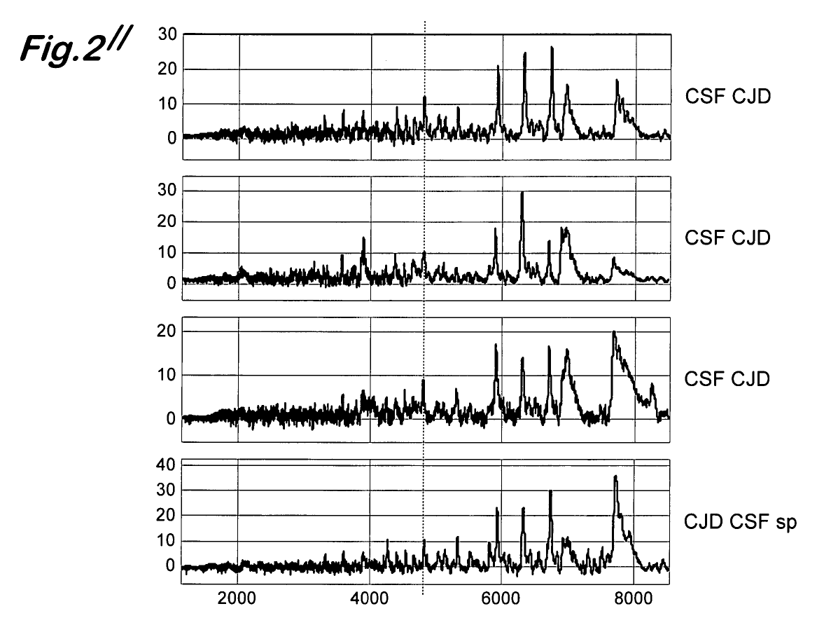

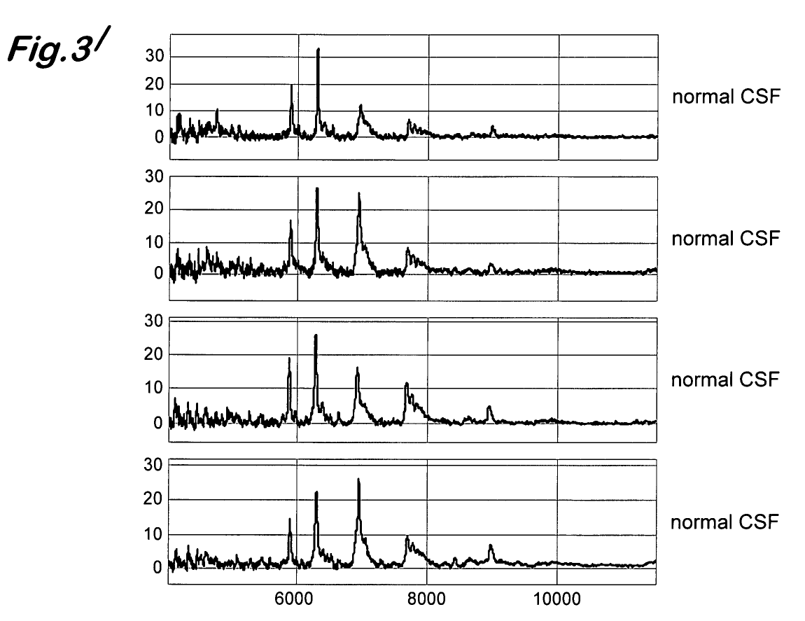

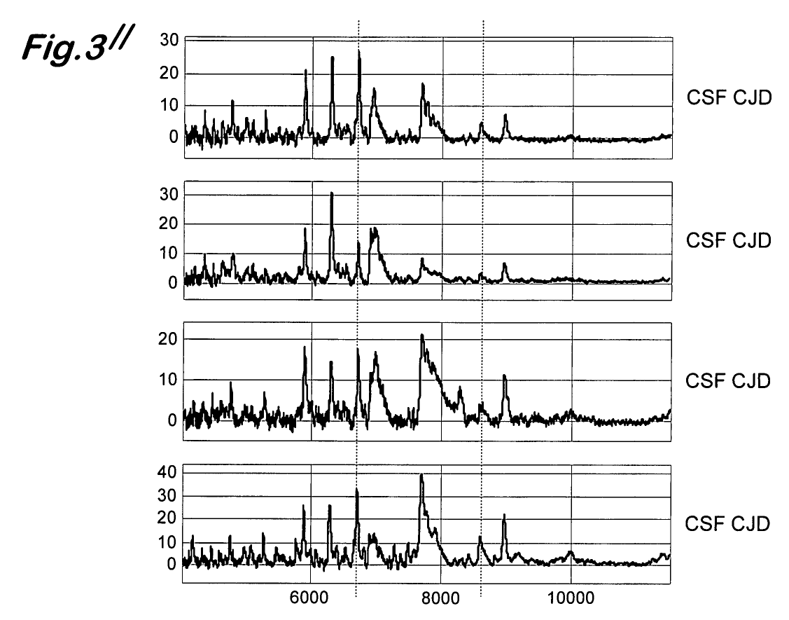

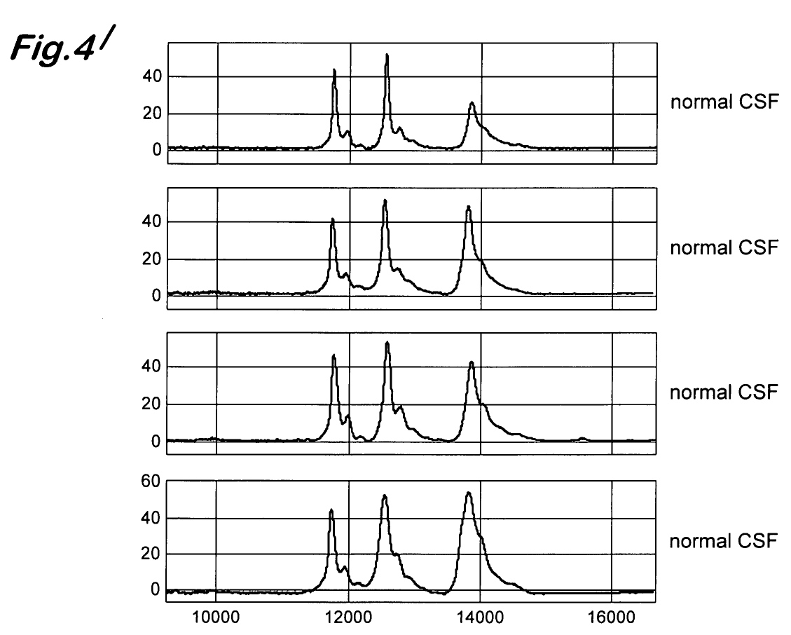

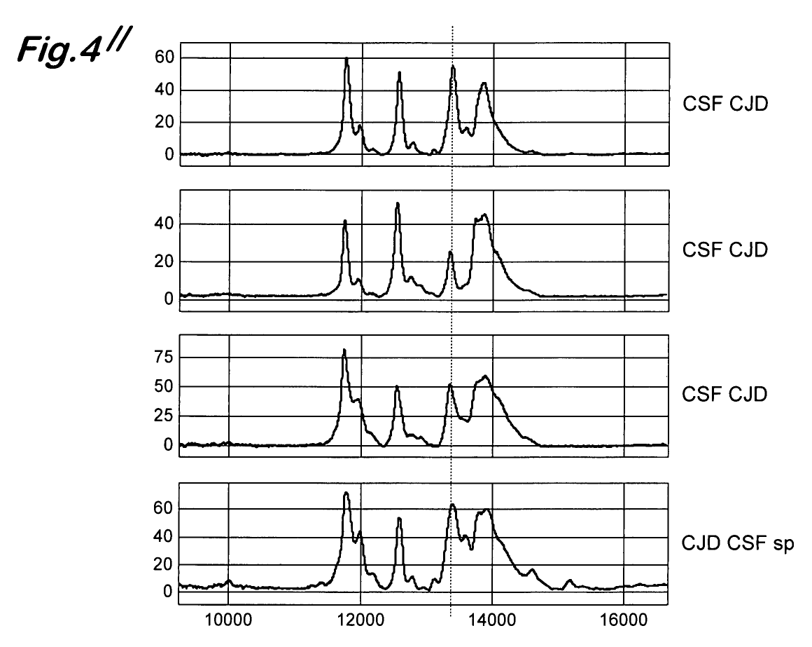

FIGS. 1 to 4 show the results of a comparative study which has been undertaken between CSF from CJD-diagnosed patients and normal CSF, using the IMAC 3 protein chip array prepared as described above. In this study, we found that four peaks were significantly differentially increased in CSF from CJD-affected patients. Their molecular weights are respectively about 4780, 6700, 8600 and 13375 (mass accuracy is around 0.1%). FIG. 1 shows two spectral views, respectively, of the normal and CJD sample, from 0 to 100,000 Da. FIG. 2 shows the protein peak of 4780 Da, FIG. 3 shows the protein peaks of 6700 and 8600 Da, and FIG. 4 shows the protein peak of 13375 Da. These data demonstrate that the peaks of about 4780, 6700, 8600 and 13375 Da can be used to diagnose CJD in CSF samples.

### EXAMPLE 2

Polypeptides in Plasma Samples from BSE-Infected Cattle and Non-Infected Cattle

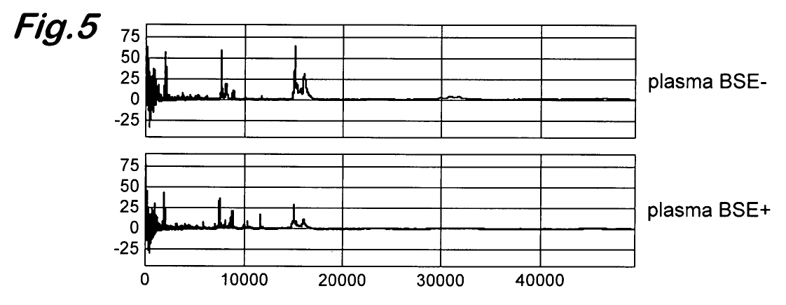

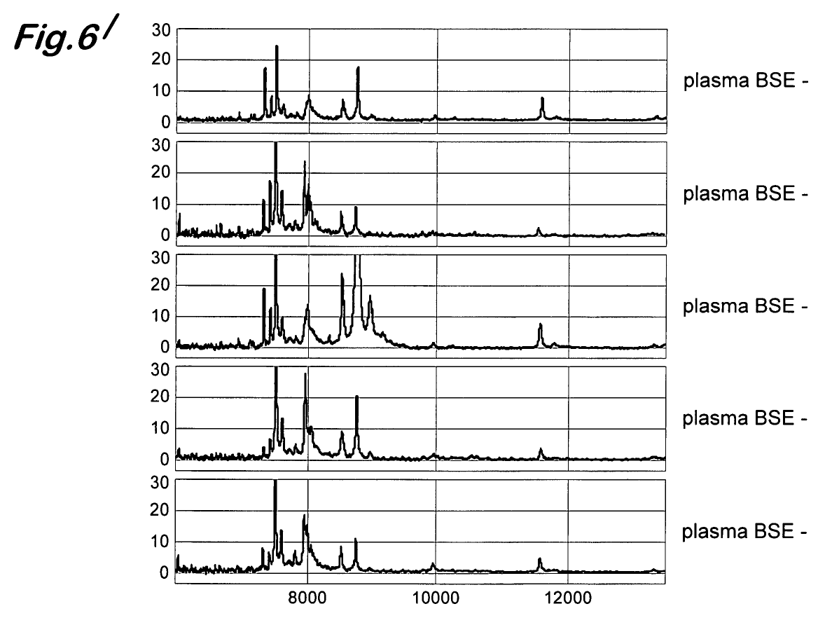

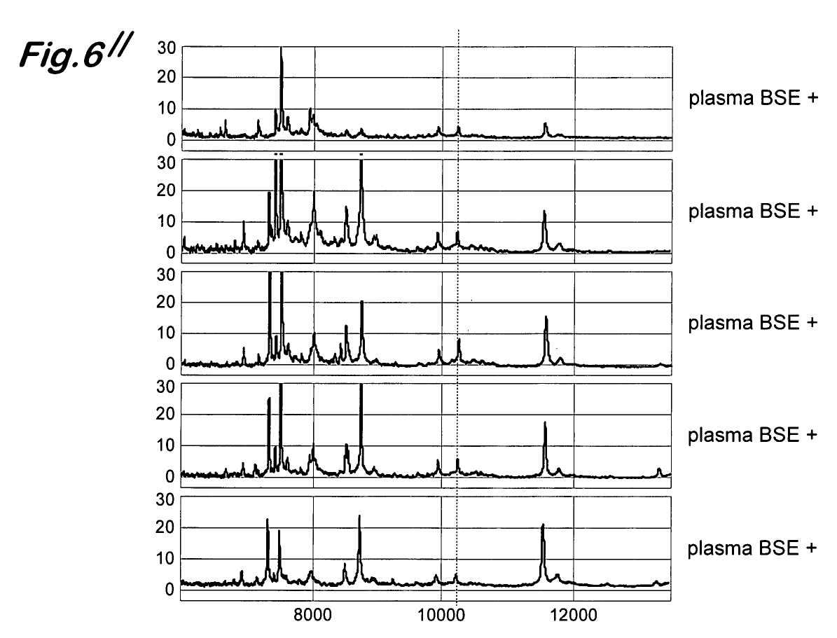

Example 1 was repeated using plasma samples from BSE-infected cattle (BSE+) and non-infected cattle (BSE−). The results are shown in FIGS. 5 and 6. FIG. 5 shows a spectral view of each kind of sample from 0 to 50,000 Da. We observed that a protein around 10220 Da was significantly increased in BSE+plasma samples, as illustrated in FIG. 6. This demonstrates that the peak of about 10220 Da can be used to diagnose BSE in plasma samples.

### EXAMPLE 3

Polypeptides in Plasma Samples from CJD-Infected Patients and Non-Infected Patients

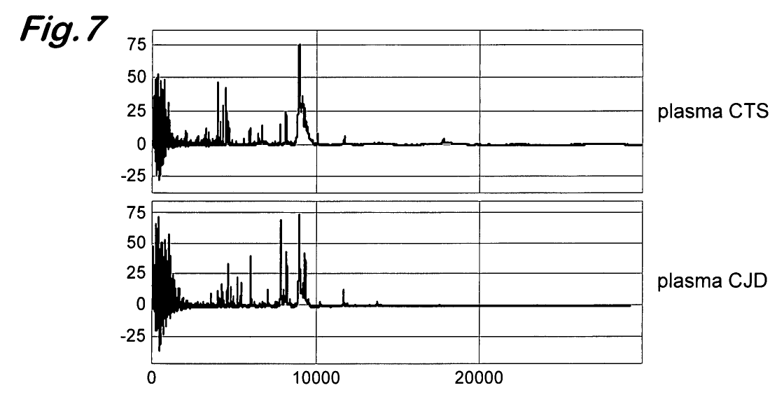

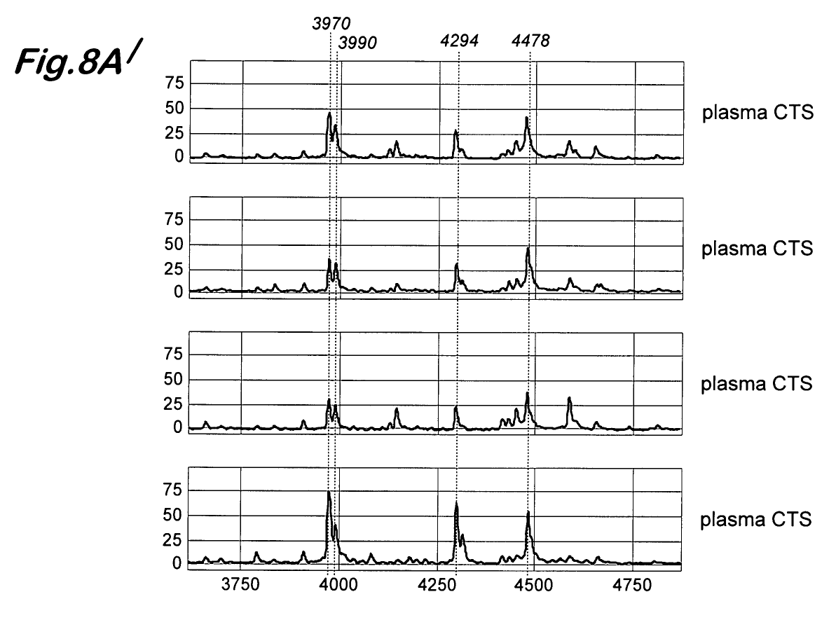

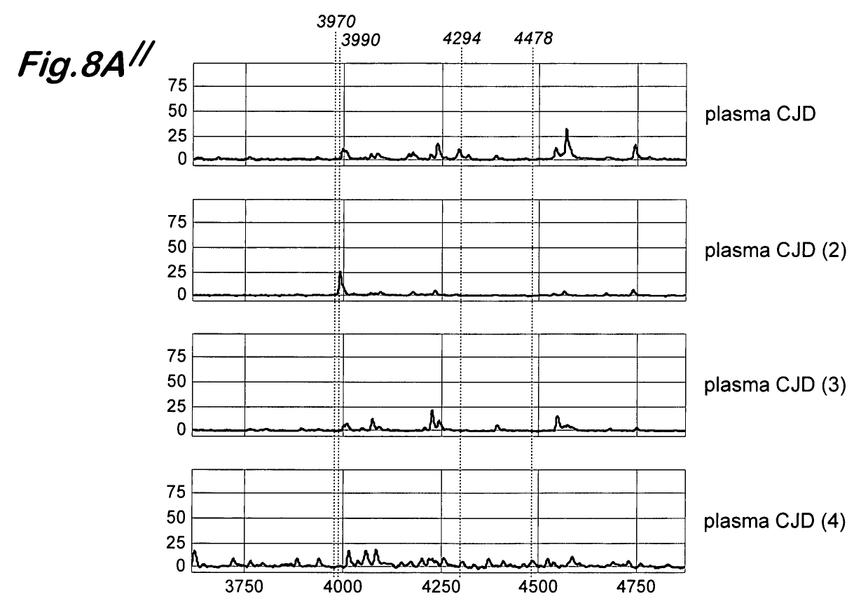

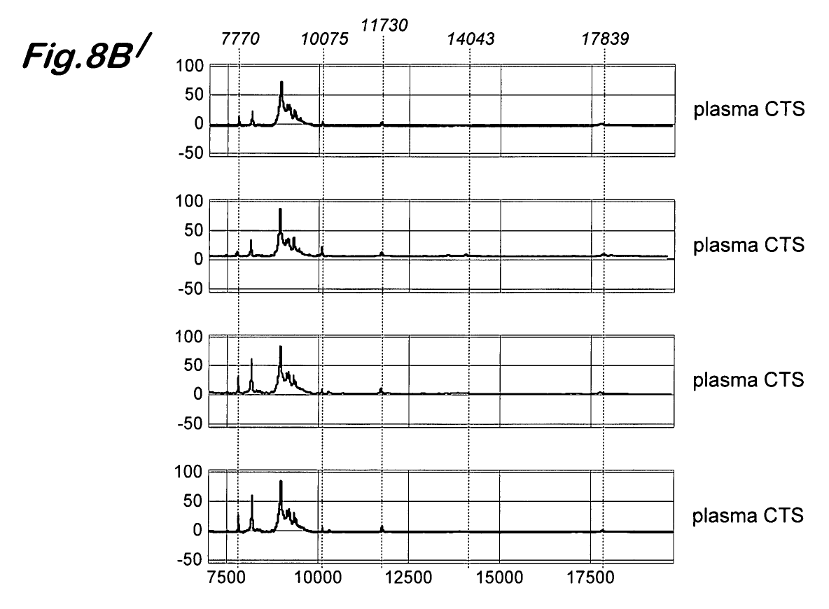

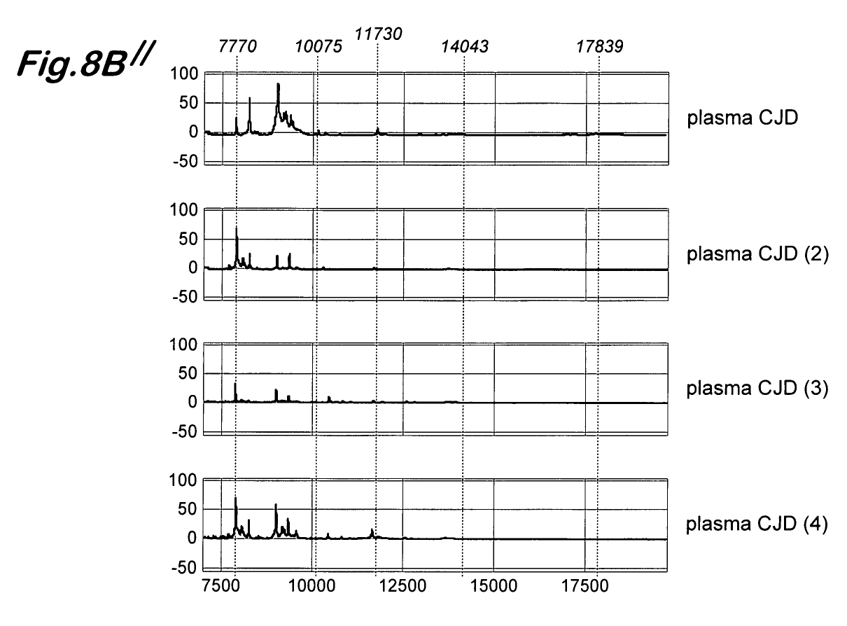

Example 2 was repeated using plasma samples from CJD-infected patients (CJD+) and non-infected patients (CJD-, also referred to as CTS=Swiss Transfusion Centre). The results are shown in FIGS. 7 and 8. FIG. 7 shows a spectral view of each kind of sample from 0 to 50,000 Da. We observed that polypeptides of about 3970, about 3990, about 4294, about 4478, about 10075, about 11730, about 14043 or about 17839 were significantly decreased in CJD+plasma samples, as illustrated in FIGS. 8A and B. We also observed that a peak of about 7770 Da was increased in CJD+plasma samples, as illustrated in FIG. 8B. This demonstrates that the peak of about 3970, about 3990, about 4294, about 4478, about 10075, about 11730, about 14043, about 17839 or about 7770 Da can be used to diagnose CJD in plasma samples.

### EXAMPLE 4

Polypeptides in Plasma Samples from CJD-Infected Patients and Non-Infected Patients

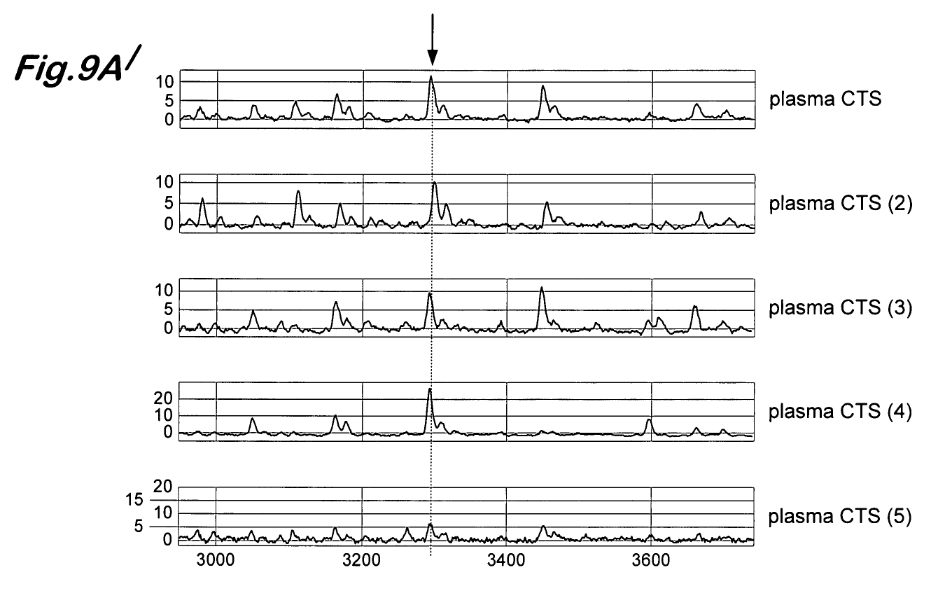

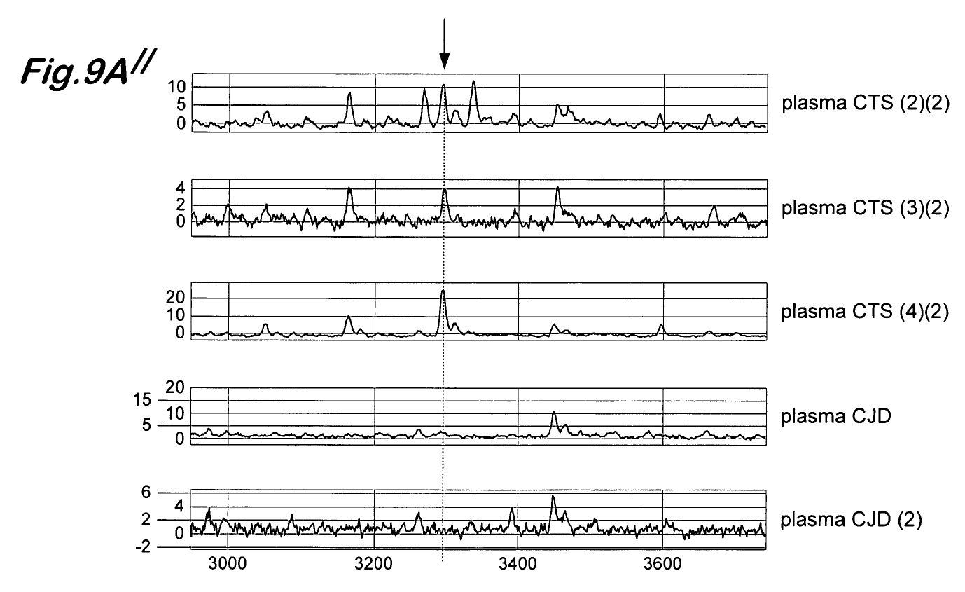

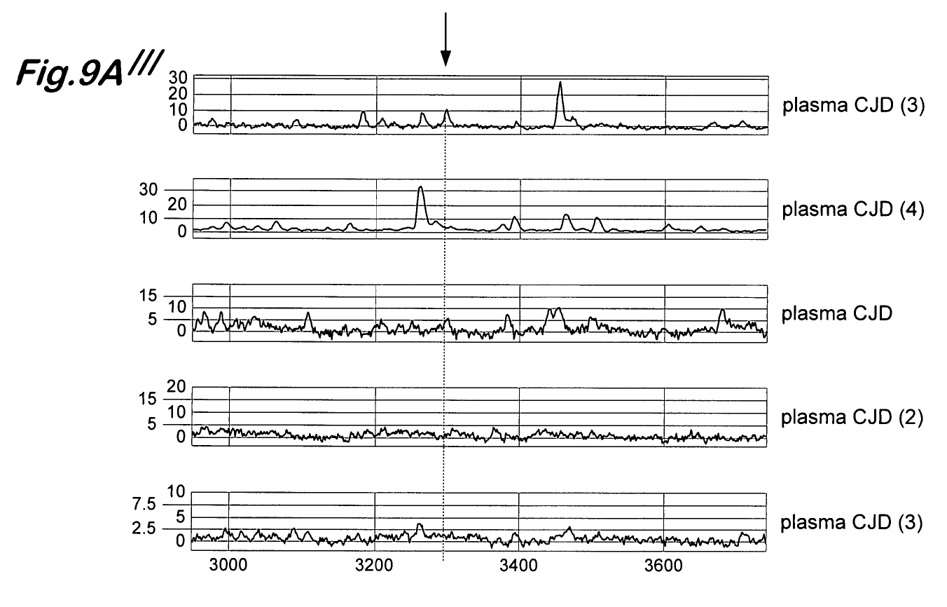

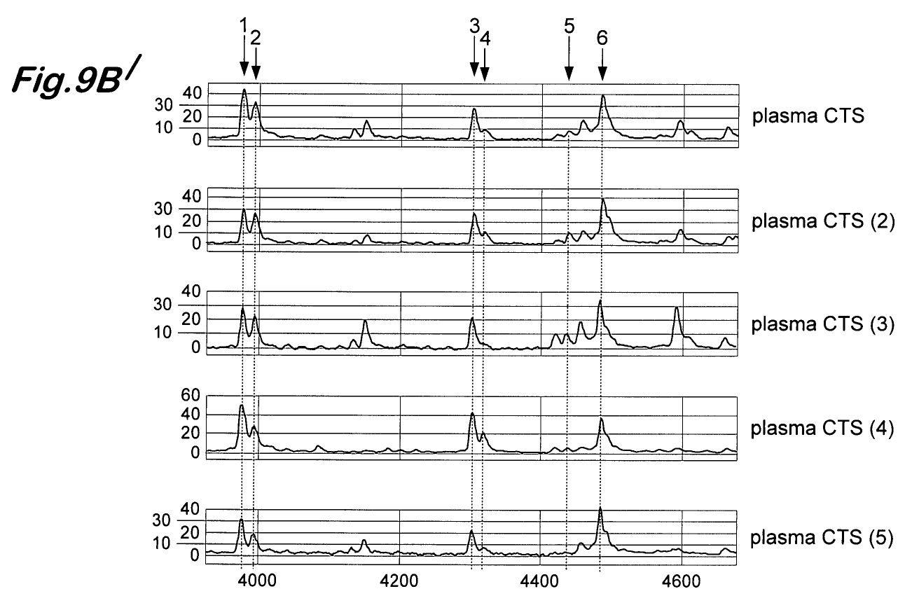

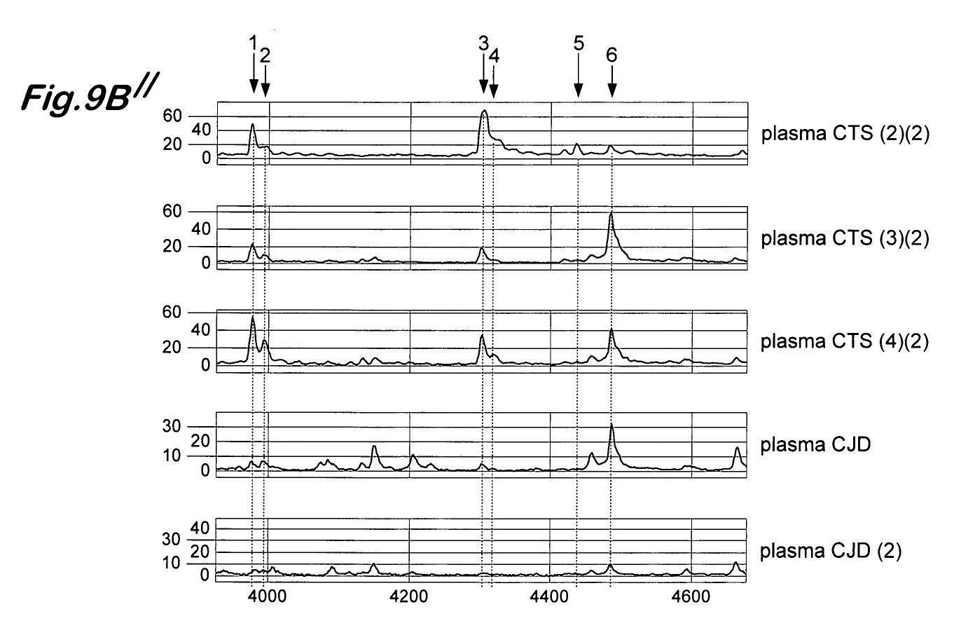

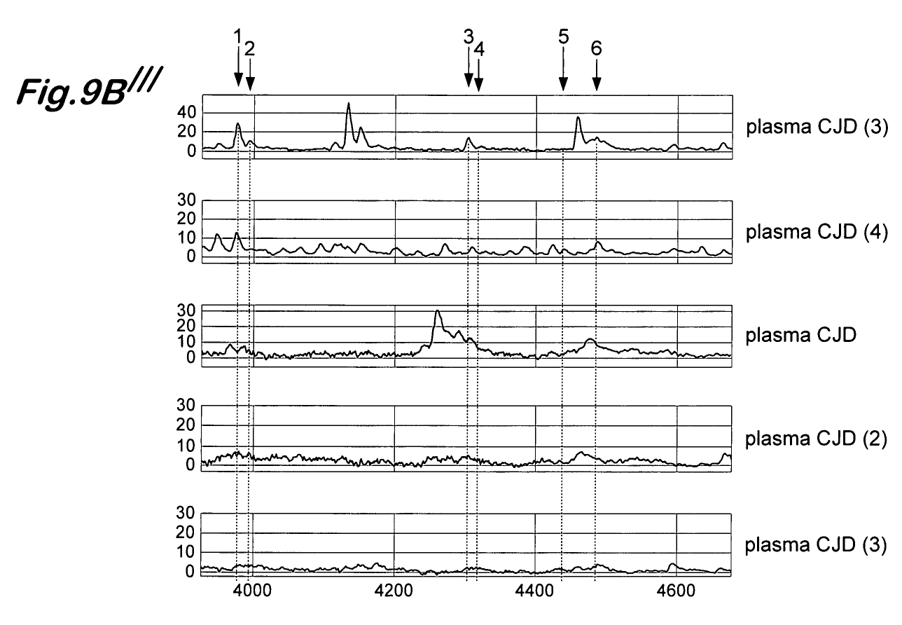

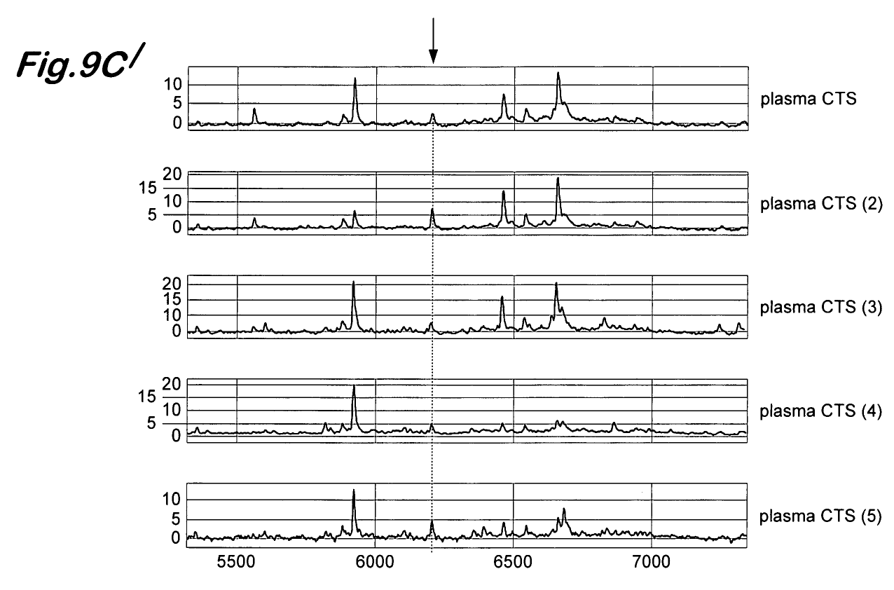

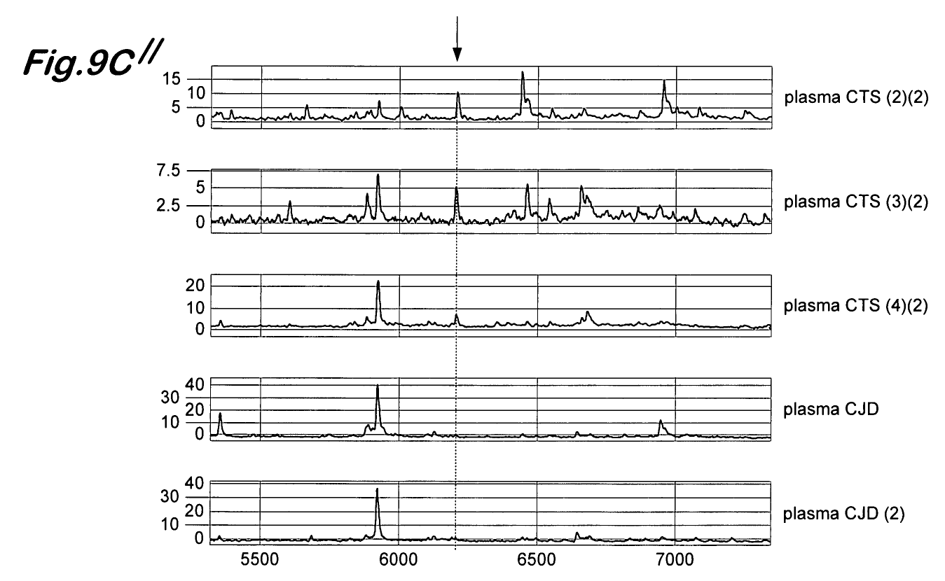

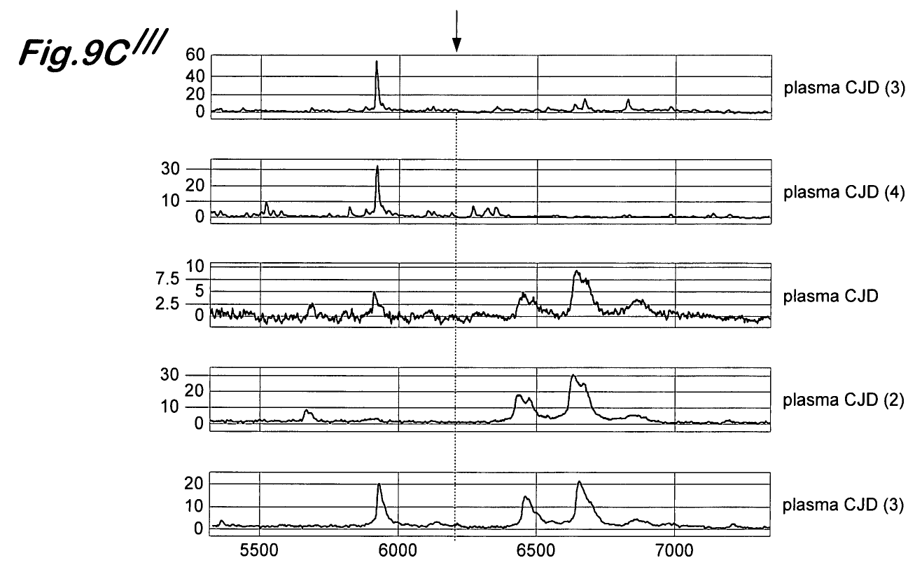

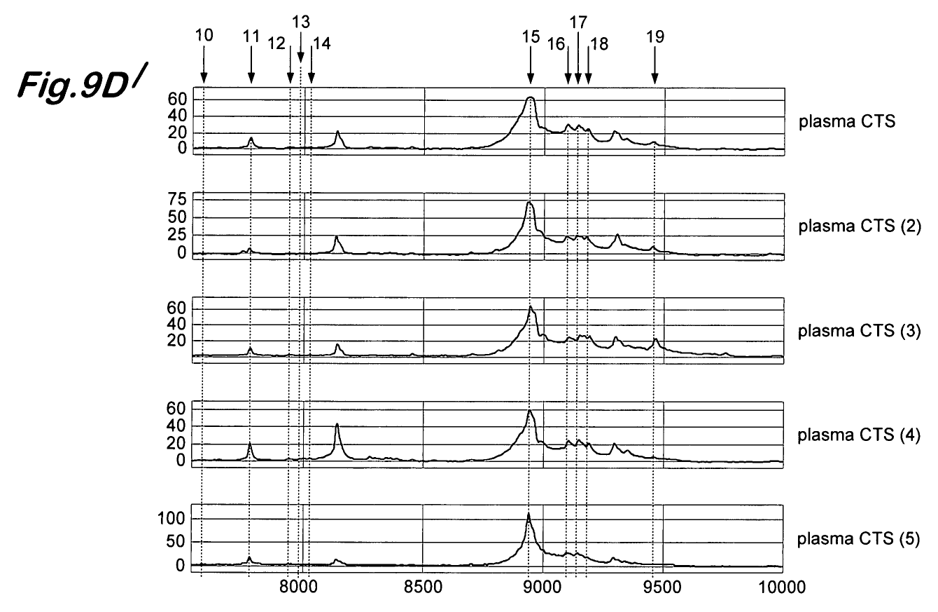

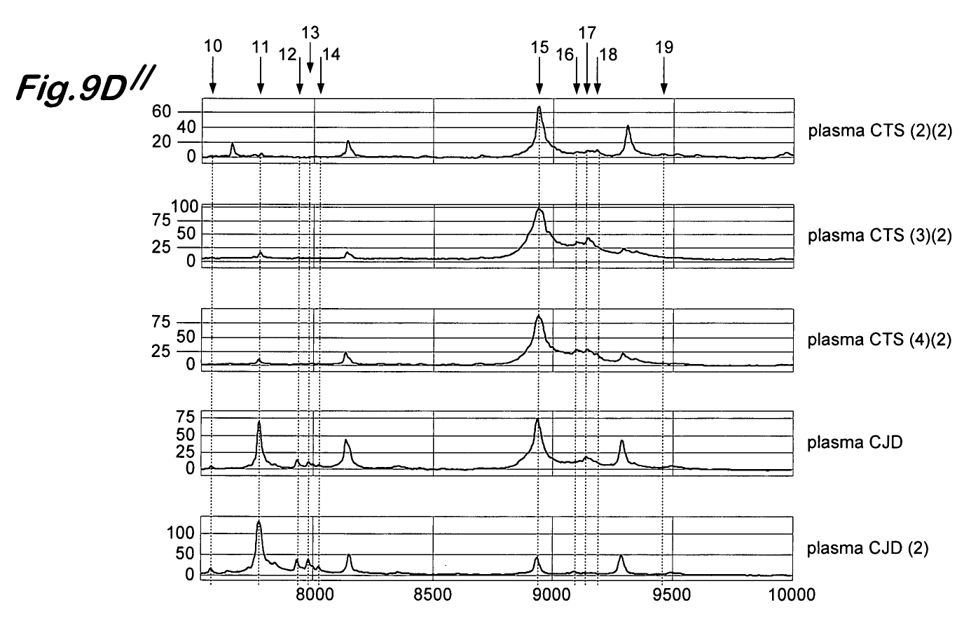

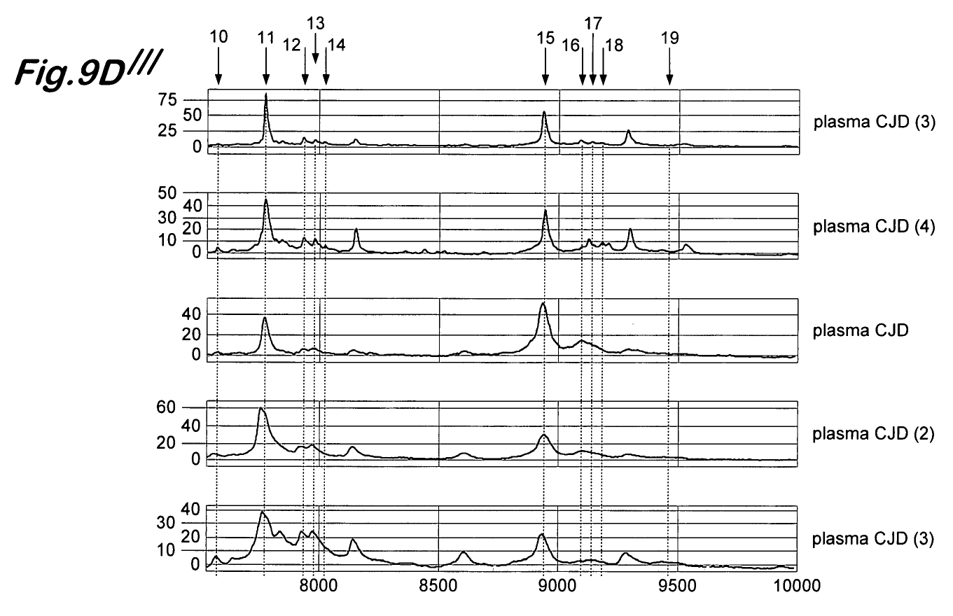

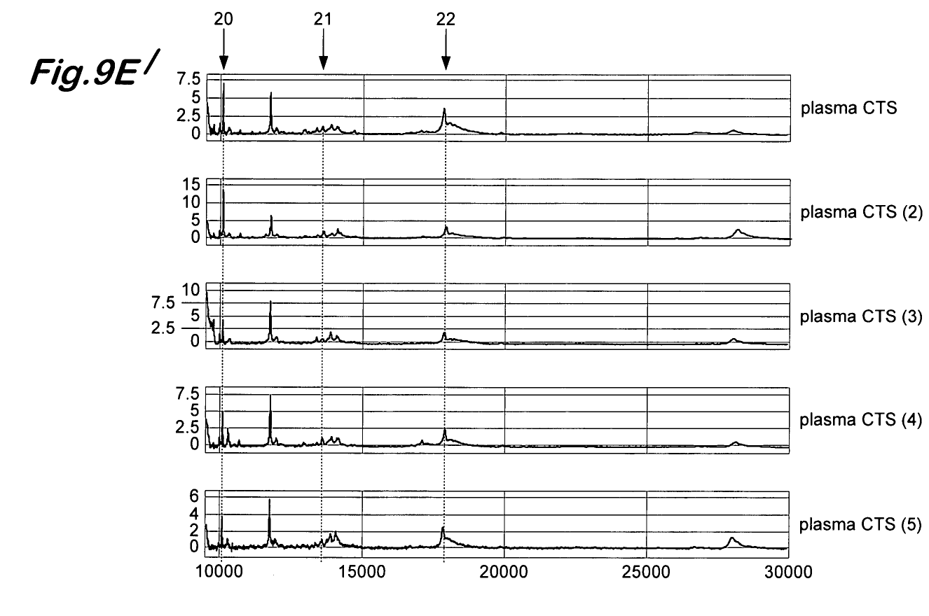

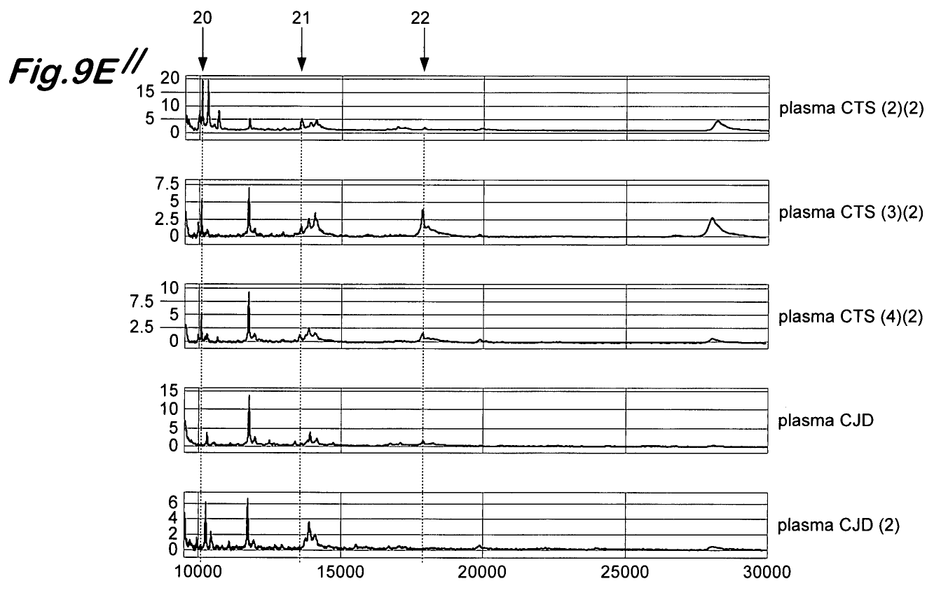

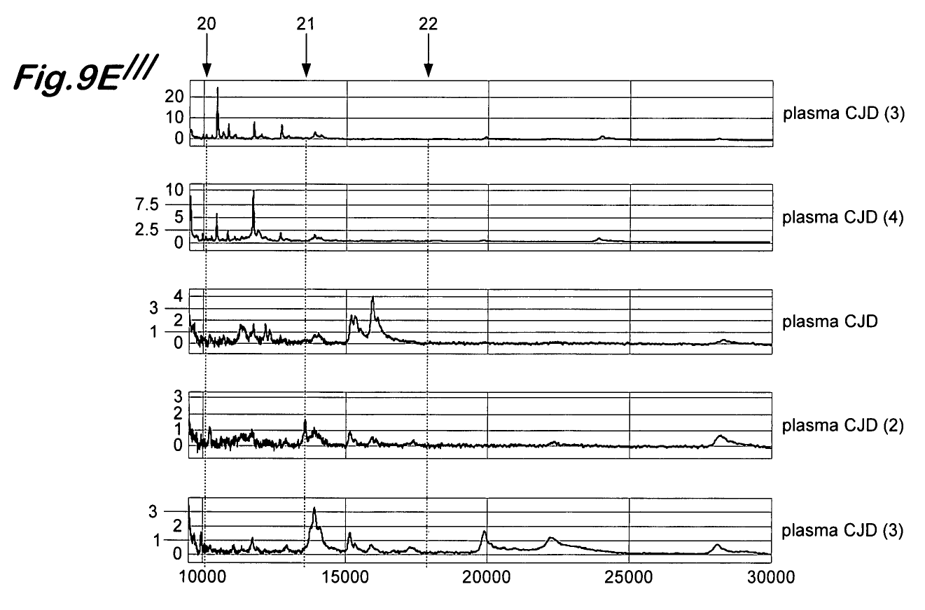

Example 3 was repeated, but using a more recent version of the software to analyse the data. The results are shown in FIGS. 9A to 9E, and indicate some new variations in protein levels, in addition to those identified in preceding Examples.

In FIG. 9A, the arrow indicates a peak at about 3295 Da, which is decreased in the CJD samples.

In FIG. 9B, the arrows in order from the left-hand side show the following:

1—a peak at about 3976 Da, which is decreased in the CJD samples (corresponding to the 3970 Da peak in Example 3)

2—a peak at about 3992 Da, which is decreased in the CJD samples (corresponding to the 3990 Da peak in Example 3)

3—a peak at about 4300 Da, which is decreased in the CJD samples (corresponding to the 4294 Da peak in Example 3)

4—a peak at about 4315 Da, which is decreased in the CJD samples

5—a peak at about 4436 Da, which is decreased in the CJD samples

6—a peak at about 4484 Da, which is decreased in the CJD samples (corresponding to the 4478 Da peak in Example 3)

In FIG. 9C, the arrow indicates a peak at about 6200 Da, which is decreased in the CJD samples.

In FIG. 9D, the arrows in order from the left-hand side show the following:

10—a peak at about 7574 Da, which is increased in the CJD samples

11—a peak at about 7773 Da, which is increased in the CJD samples (corresponding to the 7770 Da peak in Example 3)

12—a peak at about 7930 Da, which is increased in the CJD samples

13—a peak at about 7975 Da, which is increased in the CJD samples

14—a peak at about 8020 Da, which is increased in the CJD samples

15—a peak at about 8936 Da, which is decreased in the CJD samples

16—a peak at about 9107 Da, which is decreased in the CJD samples

17—a peak at about 9145 Da, which is decreased in the CJD samples

18—a peak at about 9185 Da, which is decreased in the CJD samples

19—a peak at about 9454 Da, which is decreased in the CJD samples

In FIG. 9E, the arrows in order from the left-hand side show the following:

20—a peak at about 10068 Da, which is decreased in the CJD samples (corresponding to the 10075 Da peak in Example 3)

21—a peak at about 13550 Da, which is decreased in the CJD samples

22—a peak at about 17809 Da, which is decreased in the CJD samples (corresponding to the 17839 Da peak in Example 3)

This Example demonstrates that any one of the above peaks, or more than one of them in any combination, can be used to diagnose CJD.

### EXAMPLE 5

Polypeptides in Body Fluids (Cerebrospinal Fluid, Plasma and Others) of BSE−Affected Cattle

The objective of the present study was to detect specific polypeptides in body fluids (cerebrospinal fluid, plasma and others) of BSE−affected cattle. Samples were analyzed by the Surface Enhanced Laser Desorption Ionization (SELDI) Mass Spectroscopy (MS) technology. This technology encompasses micro-scale affinity capture of proteins by using different types of retentate chromatography and then analysis by time of flight mass spectrometry. Different maps are thus generated each corresponding to a typical protein profiling of given samples that were analyzed with a Ciphergen Biosystem PBS II mass spectrometer (Freemont, Calif., USA). Differential expressed peaks were identified when comparing spectra generated in a group of plasma samples from BSE-affected cattle with a group of healthy cattle using protein chip arrays.

The SELDI analysis was performed using 2 μl of crude bovine plasma samples in order to detect specific polypeptides with metal affinity. An immobilized copper affinity array (IMAC-Cu⁺⁺) was employed in this approach to capture proteins with affinity for copper to select for a specific subset of proteins from the samples. It will be appreciated that other protein chip arrays and immobilized metal chip arrays may be substituted for the IMAC-Cu⁺⁺ affinity array. Captured proteins were directly detected using the PBSII Protein Chip Array reader (Ciphergen Biosystems, Freemont, Calif., USA).

The following protocol was used for the processing and analysis of ProteinChip arrays using Chromatographic TED-Cu(II) adsorbent array. TED is a (tris(carboxymethyl) ethylenediamine-Cu) adsorbent coated on a silicon oxide-coated stainless steel substrate.

The surface was first loaded with 10 μl of 100 mM copper sulfate to each spot and incubated for 15 minutes in a wet chamber.

The chip was thereafter washed by two quick rinses with deionized water for about 10 seconds to remove the excess unbound copper.

Before loading the samples, the I-MAC 3 array was equilibrated once with 5 μl of PBS NaCl 0.5 M for 5 minutes.

After removing the equilibration buffer, 3 μl of the same buffer were added before applying 211 of plasma. The chip was incubated for 20 minutes in a wet chamber.

The samples were thereafter removed and the surface was washed three times with the equilibration buffer (5 minutes each).

Two quick final rinses with water were performed.

The surface was allowed to air dry, followed by the addition of 0.5 μl of saturated sinapinic acid (SPA, Ciphergen Biosystem) prepared in 50% acetonitrile, 0.5% trifluoroacetic acid.

The chip was air dried again before analysis of the retained protein on each spot with laser desorption/ionization time-of-flight mass spectrometry.

The protein chip array was inserted into the instrument and analyzed once the appropriate detector sensitivity and laser energy have been established to automate the data collection.

The obtained spectra were analyzed with the Biomark Wizard software (Ciphergen Biosystems, Freemont, Calif., USA) running on a Dell Dimension 4100 PC. It generates consistent peak sets across multiple spectra.

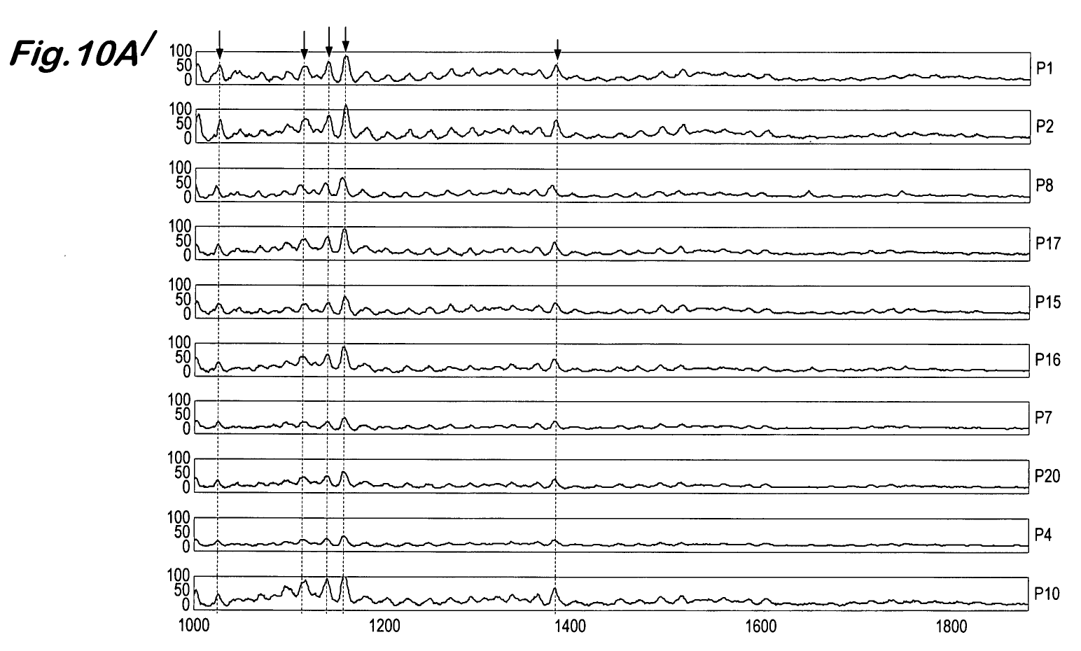

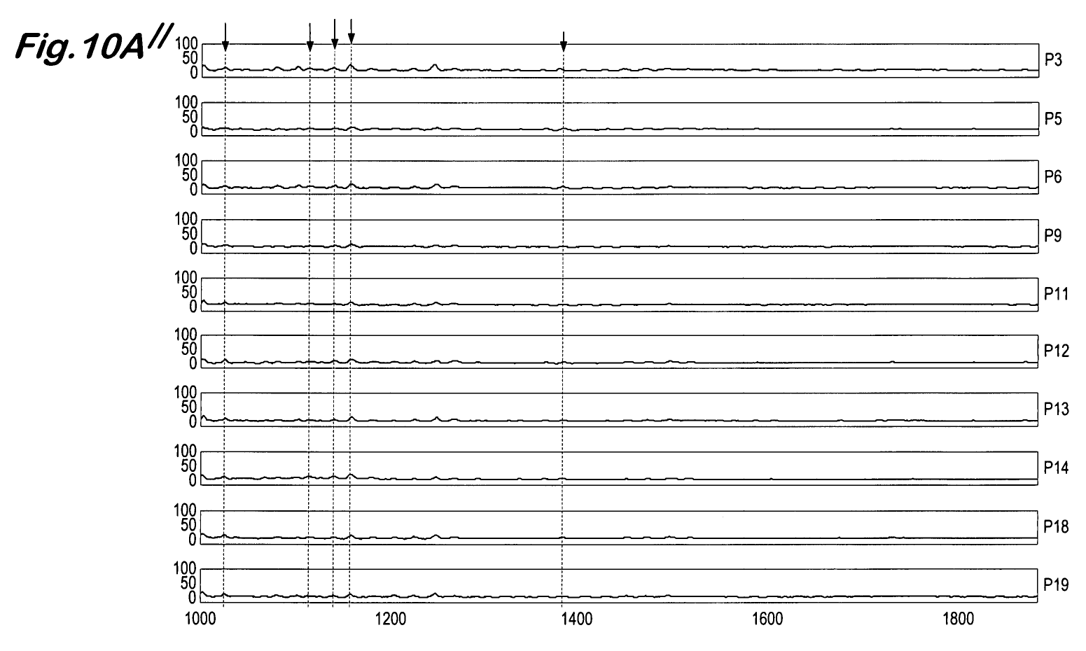

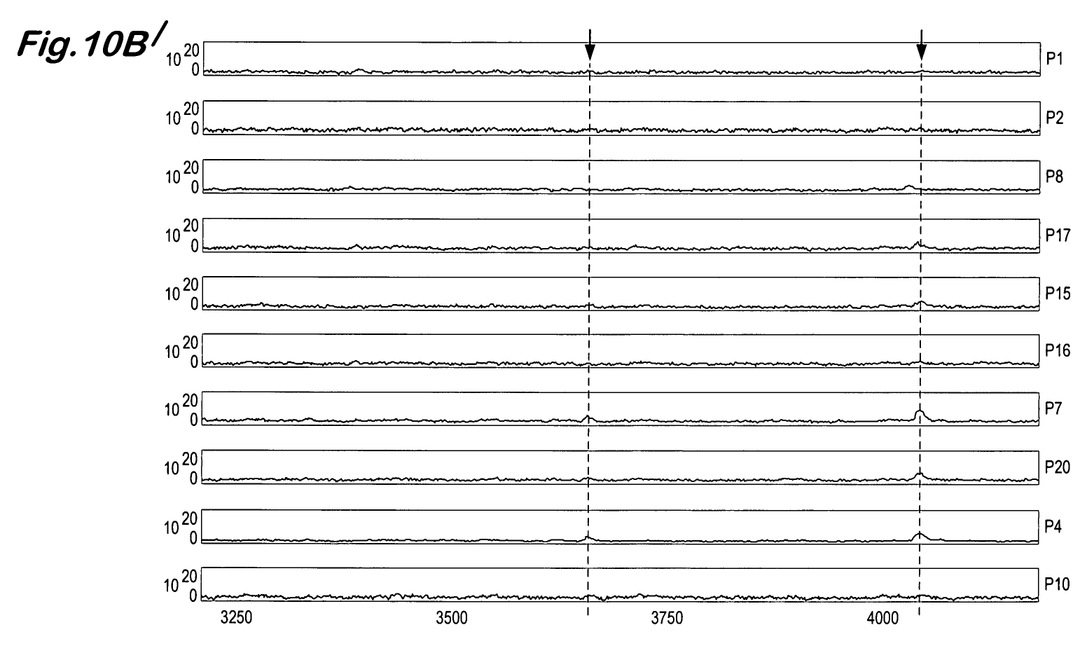

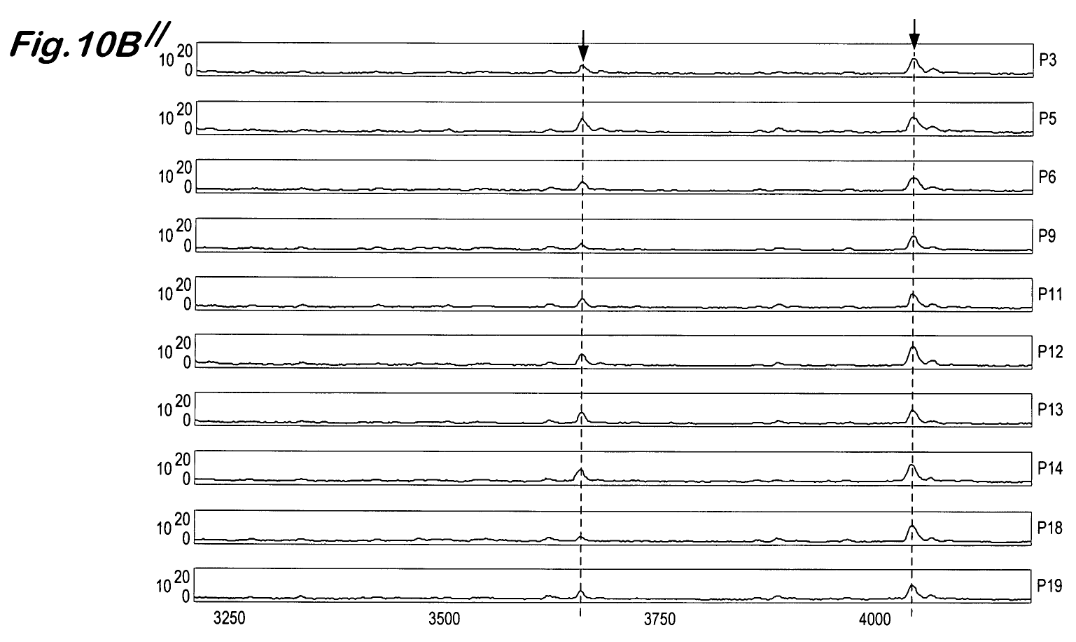

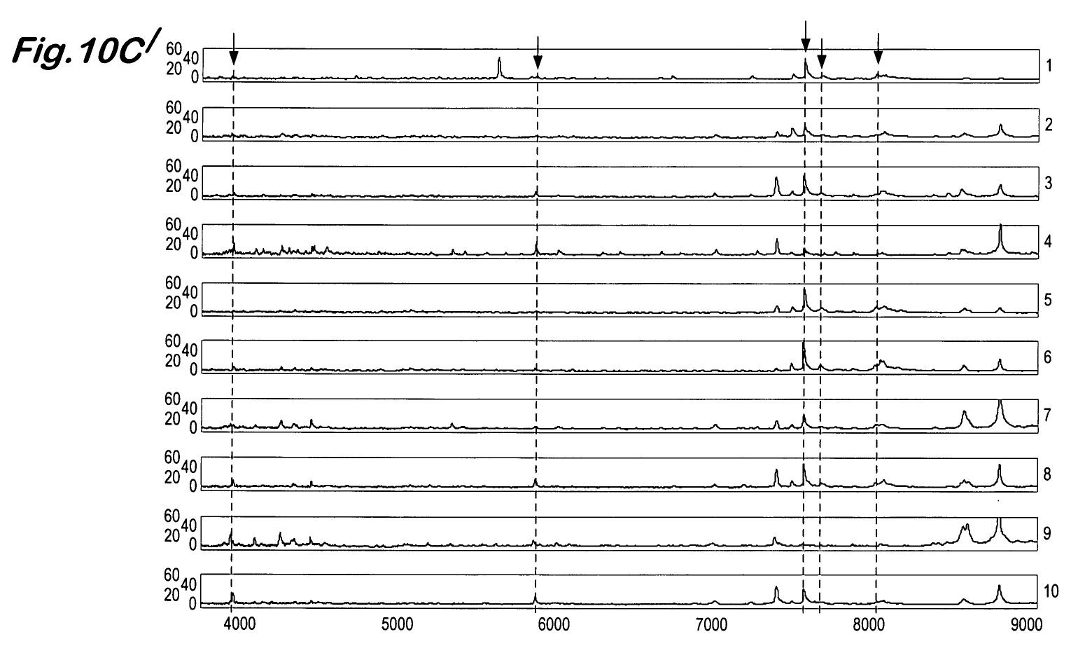

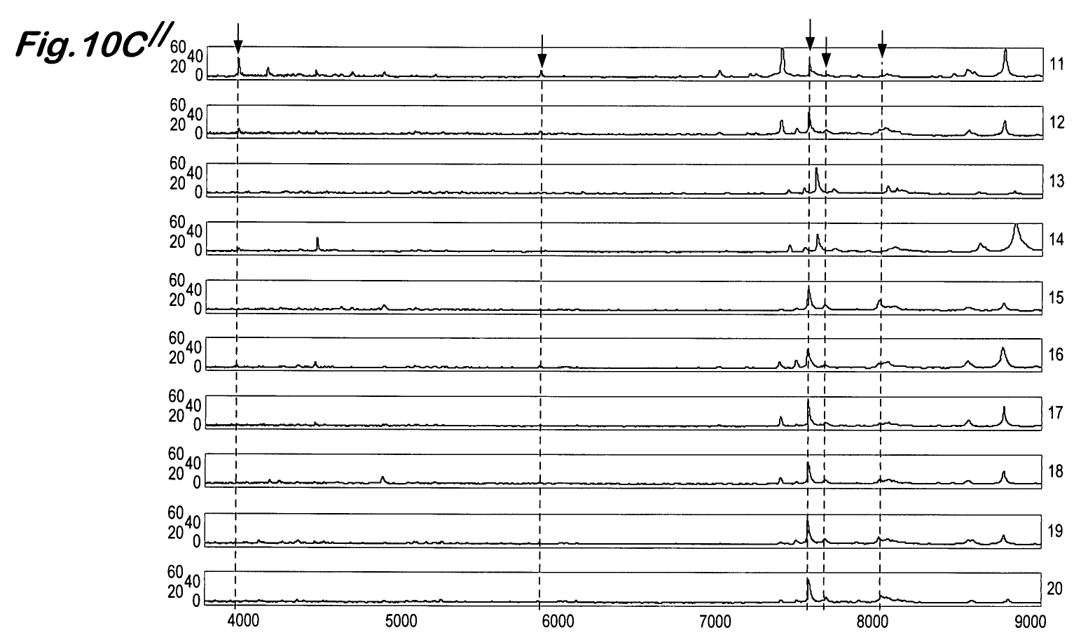

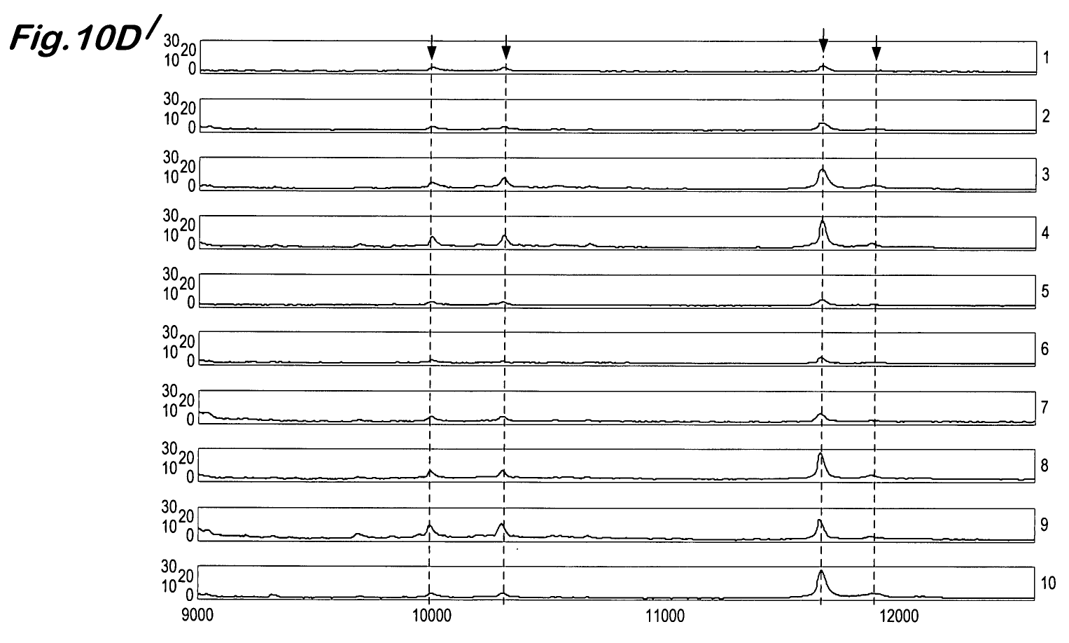

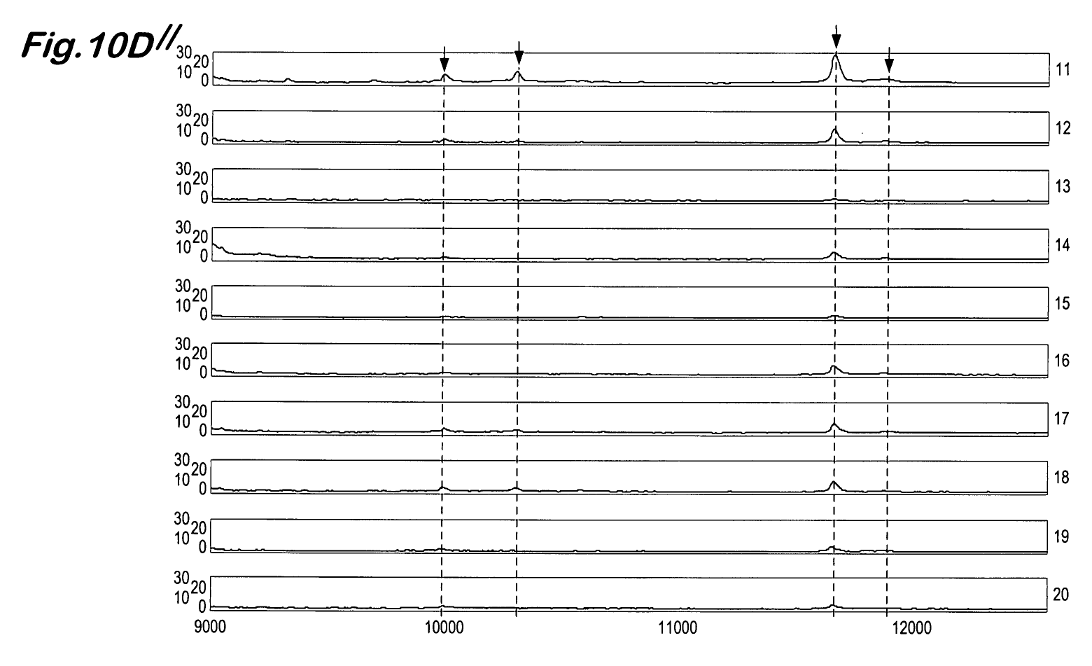

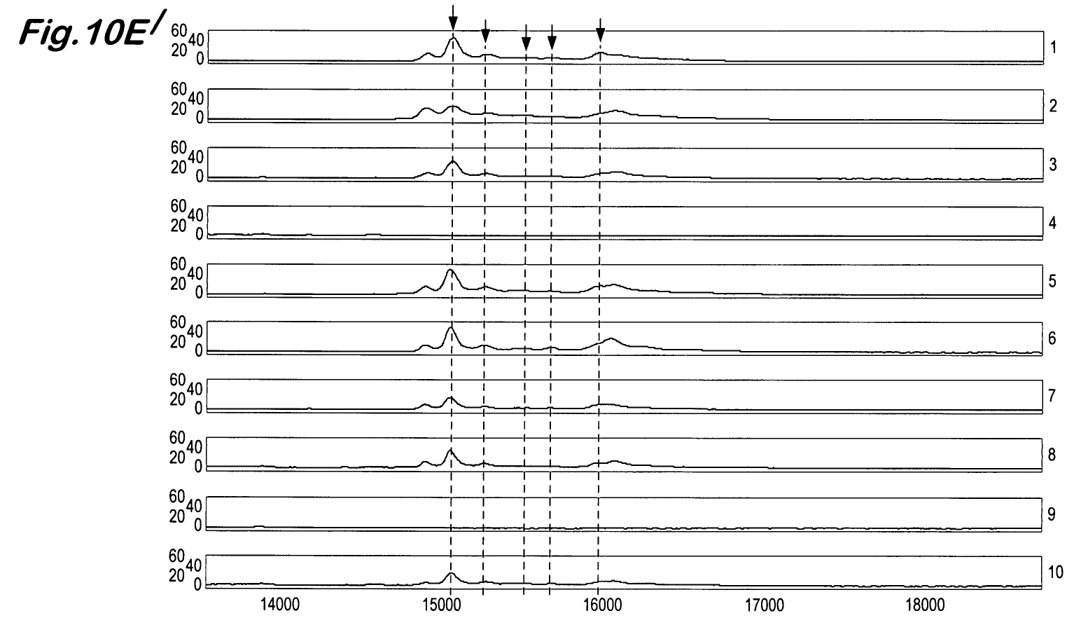

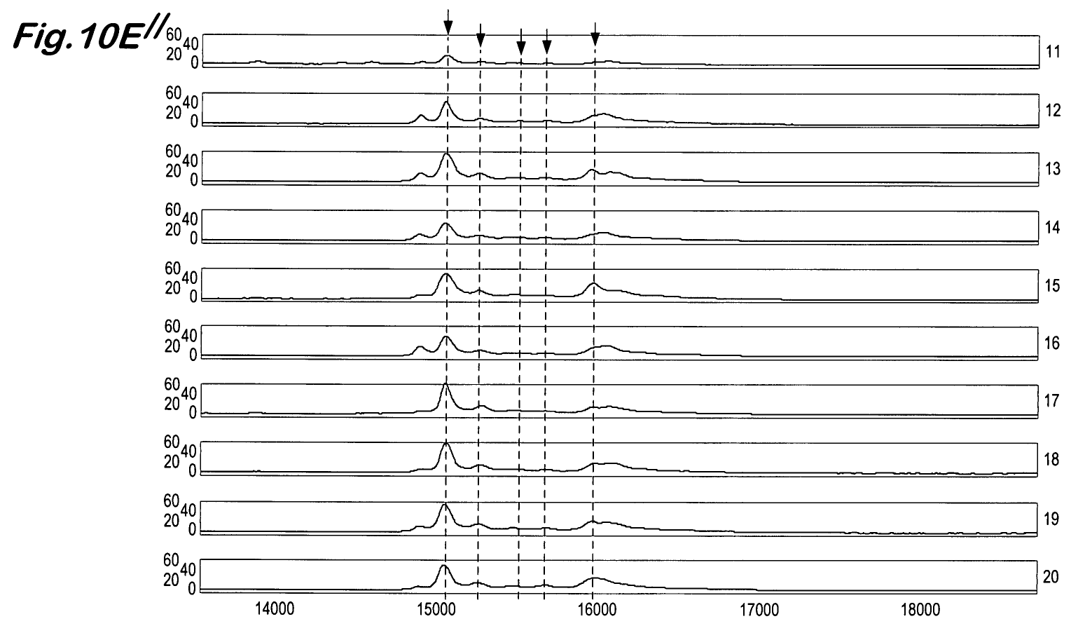

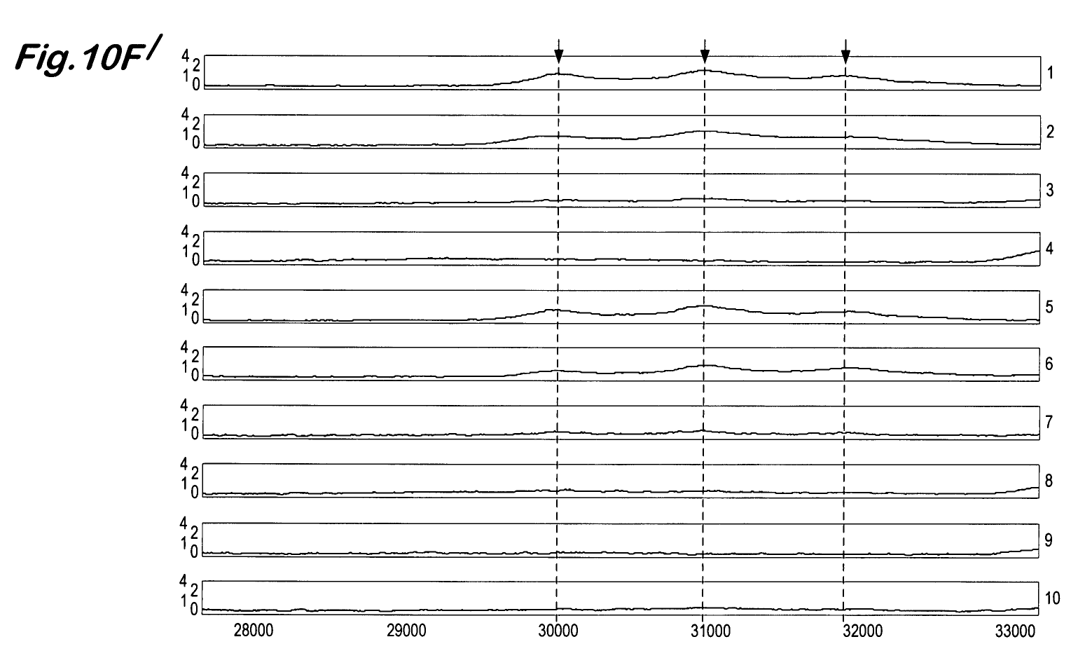

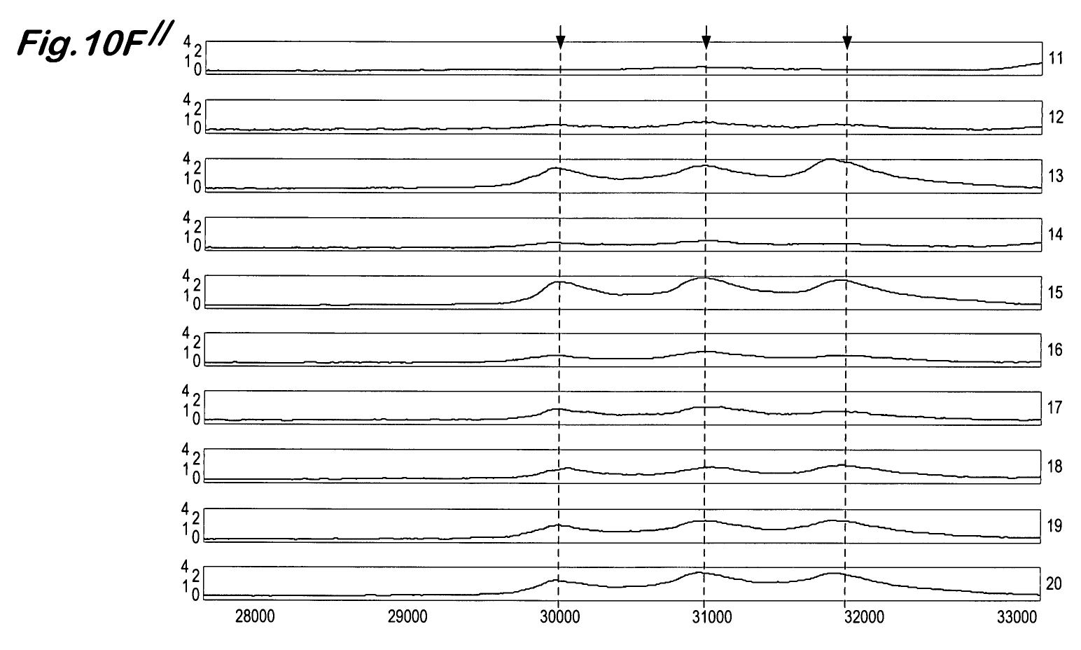

FIGS. 10A-10F show the results of a comparative study, which has been undertaken between plasma from BSE-diagnosed cattle and normal plasma, using the IMAC3 protein chip array prepared as described above. In this study, we found that 23 peaks were significantly differentially expressed in plasma from BSE-affected cattle. Their molecular weights are, respectively about 1010, 1100, 1125, 1365, 3645, 4030, 3890, 5820, 7520, 7630, 7980, 9950, 10250, 11600, 11800, 15000, 15200, 15400, 15600, 15900, 30000, 31000 and 31800 Da (mass accuracy is around 0.1%). FIG. 10 shows two spectral views, respectively, of the normal and BSE samples, from 0 to 100,000 Da. More specifically, as indicated by the vertical arrows, FIG. 10A shows the peaks at about 1010, 1100, 1125 and 1365. FIG. 10B shows the peaks at about 3645 and 4030. FIG. 10C shows the peaks at about 3890, 5820, 7520, 7630 and 7980. FIG. 10D shows the peaks at about 9950, 10250, 11600 and 11800. FIG. 10E shows the peaks at about 15000, 15200, 15400, 15600 and 15900. FIG. 10F shows the peaks at about 30000, 31000 and 31800.

Spectra P1 to P20 (FIGS. 10A-10B) correspond to a batch of samples from UK, and spectra 1 to 20 (FIGS. 10C-10F) correspond to a batch of samples from US. The status of the cattle providing the samples is indicated below in Tables 1 and 2, where negative means not affected by BSE and positive means BSE-affected cattle.

TABLE 1

| # | Type | Status |
|---|---|---|
| P1 | Plasma | Negative |
| P2 | Plasma | Negative |
| P3 | Plasma | Positive |
| P4 | Plasma | Negative |
| P5 | Plasma | Positive |
| P6 | Plasma | Positive |
| P7 | Plasma | Negative |
| P8 | Plasma | Negative |
| P9 | Plasma | Positive |
| P10 | Plasma | Negative |
| P11 | Plasma | Positive |
| P12 | Plasma | Positive |
| P13 | Plasma | Positive |
| P14 | Plasma | Positive |
| P15 | Plasma | Negative |
| P16 | Plasma | Negative |
| P17 | Plasma | Negative |
| P18 | Plasma | Positive |
| P19 | Plasma | Positive |
| P20 | Plasma | Negative |

TABLE 2

| # | Type | Status |
|---|---|---|
| 1 | Plasma | Positive |
| 2 | Plasma | Positive |
| 3 | Plasma | Positive |
| 4 | Plasma | Positive |
| 5 | Plasma | Positive |
| 6 | Plasma | Positive |
| 7 | Plasma | Positive |
| 8 | Plasma | Positive |
| 9 | Plasma | Positive |
| 10 | Plasma | Positive |
| 11 | Plasma | Positive |
| 12 | Plasma | Negative |
| 13 | Plasma | Negative |
| 14 | Plasma | Negative |
| 15 | Plasma | Negative |
| 16 | Plasma | Negative |
| 17 | Plasma | Negative |
| 18 | Plasma | Negative |
| 19 | Plasma | Negative |
| 20 | Plasma | Negative |

These data demonstrate that the peaks of about 1010, 1100, 1125, 1365, 3645, 4030, 3890, 5820, 7520, 7630, 7980, 9950, 10250, 11600, 11800, 15000, 15200, 15400, 15600, 15900, 30000, 31000 and 31800 Da can be used to diagnose BSE in plasma samples.

### EXAMPLE 6

Identification and Up-Regulation of Cystatin C in CSFs of CJD-Affected Patients

CSF samples (100 μl) obtained from the CJD surveillance unit (sporadic or definite varient of CJD, as well as Not Case) were investigated using SELDI protein Chip Array technology. WCX2 (a weak cation exchange arrray with carbohydrate functionality), SAX2 (a strong anion exchange array with quaternary amine functionality) and IMAC3 (an immobilized metal affinity capture array with nitrotriacetic acid) surfaces were used in order to investigate differential specific binding of the proteins in the samples obtained. Various comparisons were performed between firstly samples from definite and sporadic cases (CJD samples) versus corresponding controls (Not Case) in order to discriminate between samples from patients with CJD symptoms and samples from patients with similar symptoms which were not CJD (Not Case=Control). The whole groups of samples from CJD-diagnosed patients compared with controls from the institute and the inventors' 14.3.3 samples were analyzed without any distinction in the diagnosed sub-population. Comparison of these samples did not allow sporadic and/or variant CJD samples to be distinguished from their corresponding controls (Not Case).

The inventors therefore confirmed the presence of a dementia marker of molecular weight about 13365-13370 (±0.5%), which in addition to metal affinity also demonstrates cationic properties.

The 13365-13370 marker was purified by fractionating the Not Case CSF samples using anionic exchange chromatography. Spin Sax columns designed for such fractionation separate proteins according to their net charge and were used to elute proteins stepwise with decreasing pI using buffers with increasing salt concentration and decreasing p1. The protein profiles obtained using both IMAC3 and SAX chips were similar. Each fraction was loaded on a SDS PAGE for further identification of the 13350 mw peak. A preparative colloidal blue-stained Tris-Tricine 1-DE gel was run and stained with silver nitrate to permit band excision and digestion followed by mass spectrometry identification. Identification of the squared bands excised from the gel followed the MALDI-TOF, MALDI-TOF-TOF (Applied Biosystems: provides partial sequence determination using collision-induced dissociation fragment analysis) or nanoLC Q-tof approaches. The 13350 peak was identified as cystatin C.

Matrix-assisted laser desorption/ionization-time of flight mass spectrometry (MALDI-TOF MS) is a relatively novel technique in which a co-precipitate of an UV-light absorbing matrix and a biomolecule is irradiated by a nanosecond laser pulse. Most of the laser energy is absorbed by the matrix, which prevents unwanted fragmentation of the biomolecule. The ionized biomolecules are accelerated in an electric field and enter the flight tube. During the flight in this tube, different molecules are separated according to their mass to charge ratio and reach the detector at different times. In this way each molecule yields a distinct signal. The method is used for detection and characterization of biomolecules, such as proteins, peptides, oligosaccharides and oligonucleotides, with molecular masses between 400 and 350,000 Da.

In order to validate the identification and the up-regulation of cystatin C in CSFs of CJD-affected patients, the inventors performed Western blot experiments using an antibody specific for human cystatin C on 8 demented CSF samples versus 8 CSF of CJD−affected patients (3 variant and 5 sporadic CJD).

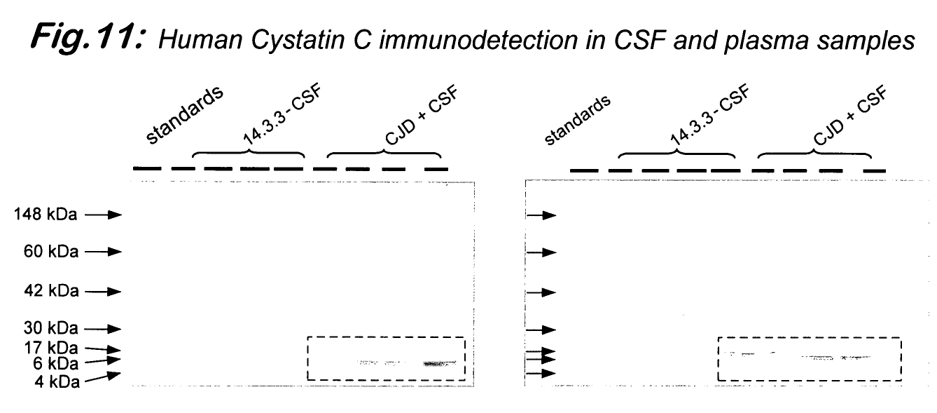

FIG. 11 shows the specific increased signal that the inventors obtained in the 8 CJD-affected patients tested in comparison to controls, showing that Cystatin provides a cerebrospinal fluid marker of CJD.

### EXAMPLE 7

Plasma of BSE Affected Cattle: SELDI Analysis.

Previous comparative studies using SELDI analysis of BSE+ and BSE−plasma samples allowed the inventors to highlight several protein clusters as being differentially expressed. One such cluster with a mass centered around 15000 Da was selected for further analysis, being differentially over-expressed in samples from BSE-affected cattle. Plasma of BSE-affected cattle was subjected to one-DE electrophoresis and the band migrating with a mass of 15000 Da as determined using standard mass marker proteins was excised, digested with trypsin and subjected to mass spectrometry by MAL:DI-TOF. By this method the 15000 Da protein species in the SELDI analysis were putatively identified as isoforms of bovine hemoglobin.

In order to investigate and validate the hemoglobin content of these samples, the inventors looked for antibodies that could cross react with bovine Hb. A goat polyclonal antibody anti-human Hb that reacts weakly with equine and bovine hemoglobin (J16, Biomeda), as well as a sheep polyclonal antibody raised against native Hb from erythrocytes expected to exhibit cross-reactivity with Hb from other species (4870-3980, Biotrend-Anawa) were tested. Experiments were carried out using as positive controls, native purified human and bovine Hb (4870-4056 and 4870-2002, Biotrend-Anawa).

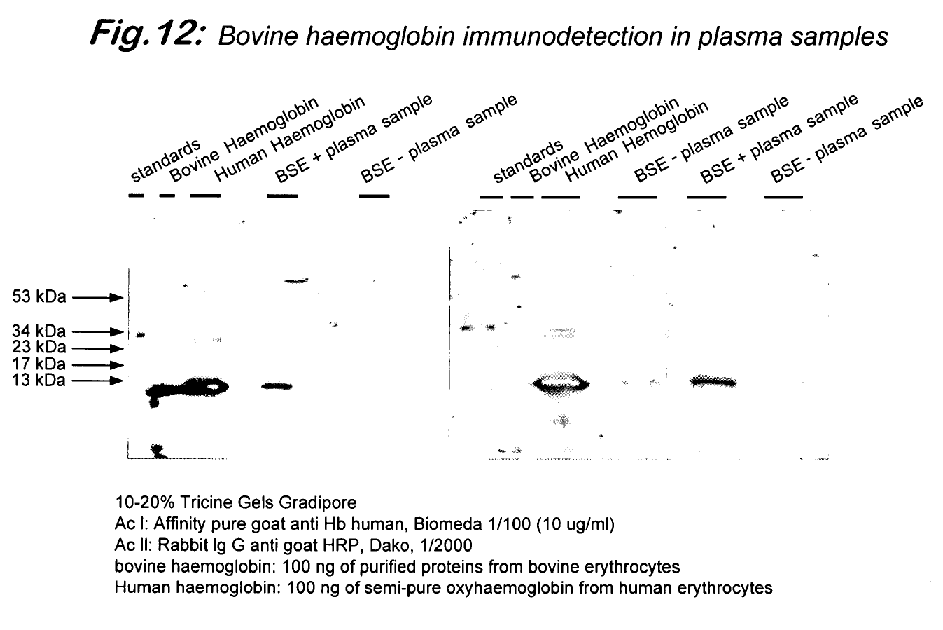

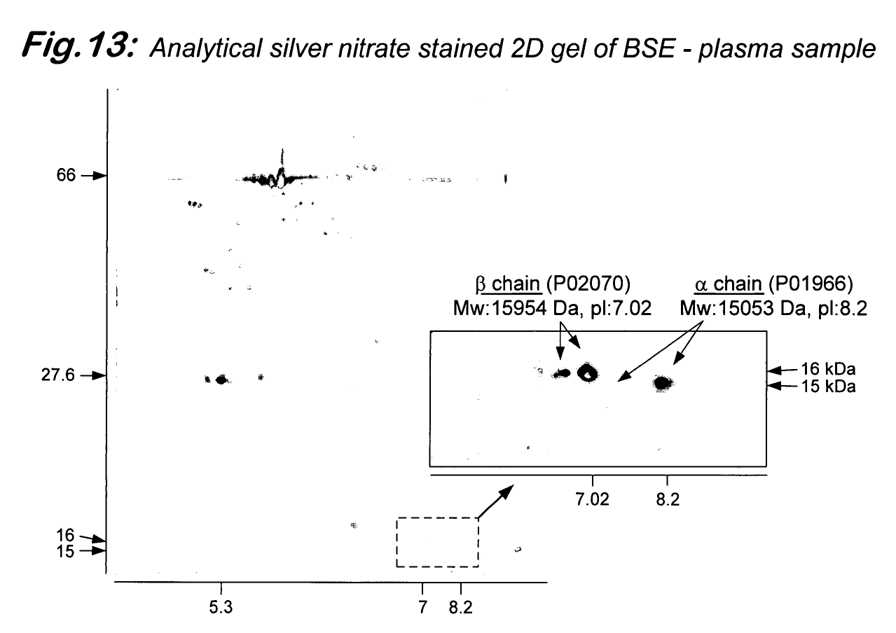

FIG. 12 highlights the Western blot experiments performed with the goat polyclonal antibody from Biomeda. Besides the strong signal obtained with human and bovine hemoglobin, an increased level of a protein at the expected size in the 3 BSE+tested plasma samples may indeed be observed. The Western blot experiments performed with the second antibody did not show any signal (data not shown). Silver stained analytical 2-DE gel (FIG. 13) and an immunodetection experiment on 2-DE PVDF membranes of BSE−plasma sample were performed, in order to see if several spots could be thus detected. The enlarged portion of FIG. 13 shows 4 spots in the expected area. Two spots seem to correspond to α chains (15053 Da, pI 8.19) and 2 others spots seem to correspond to β chains (15954 Da, pI 7.02). Knowing that the α chain has 4 putative modification sites consisting of N-glycosylation, Protein Kinase C Phosphorylation, Casein Kinase 2-Phosphorylation and N-Myristoylation, as well as β chains having similar ones with an Amidation site instead of N-glycosylation one (Scan Prosite), these explain variations in their main molecular weight, as well as the numerous shoulders highlighted in SELDI spectra.

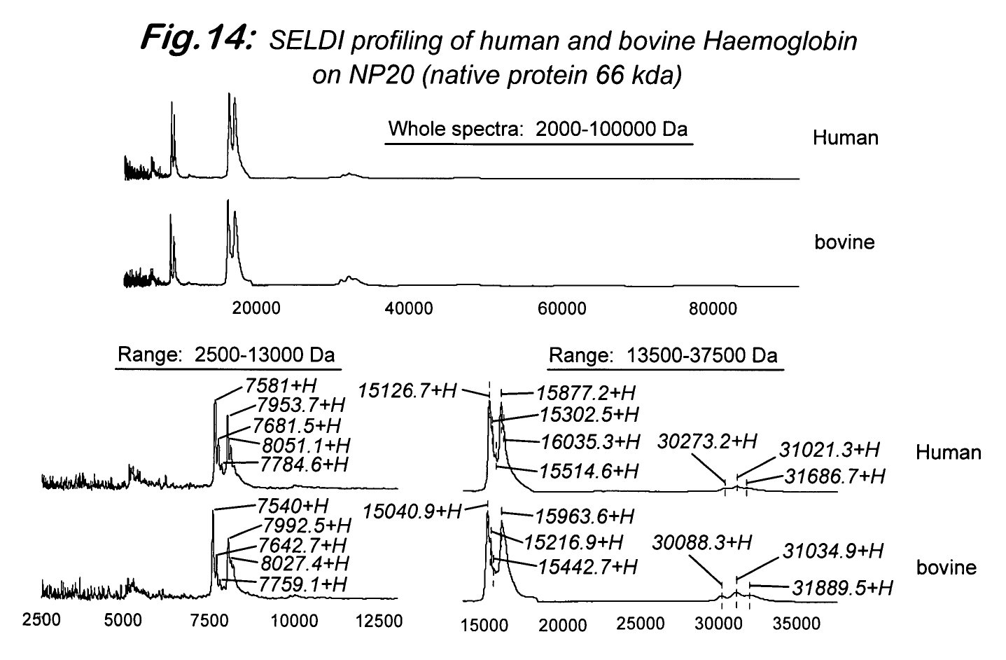

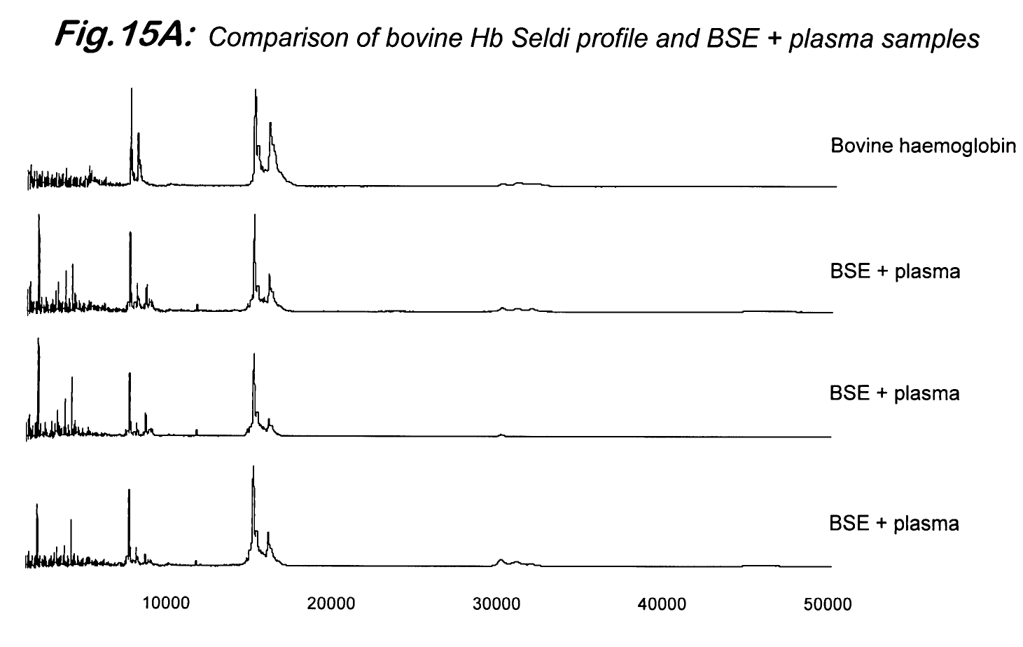

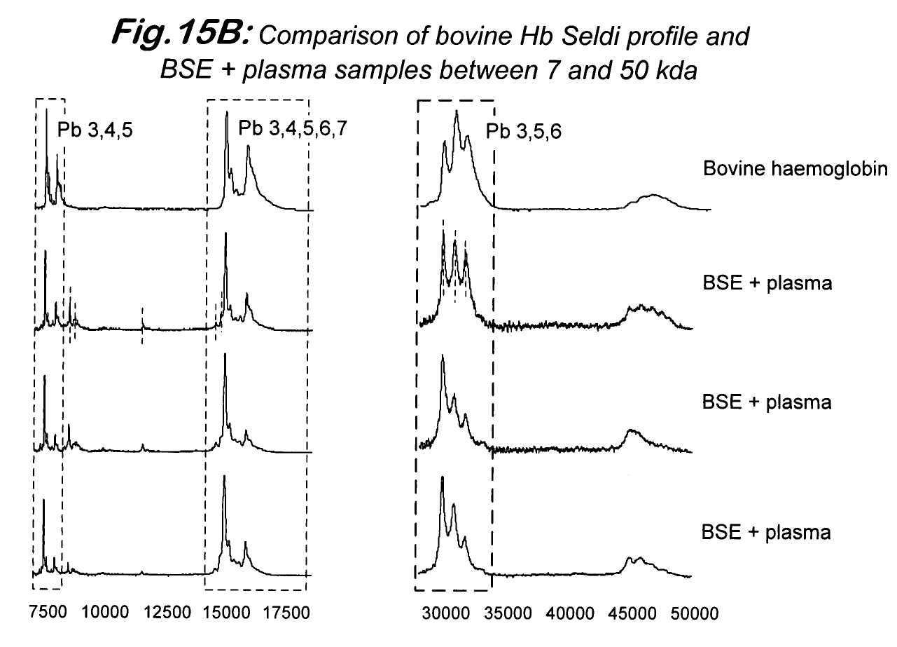

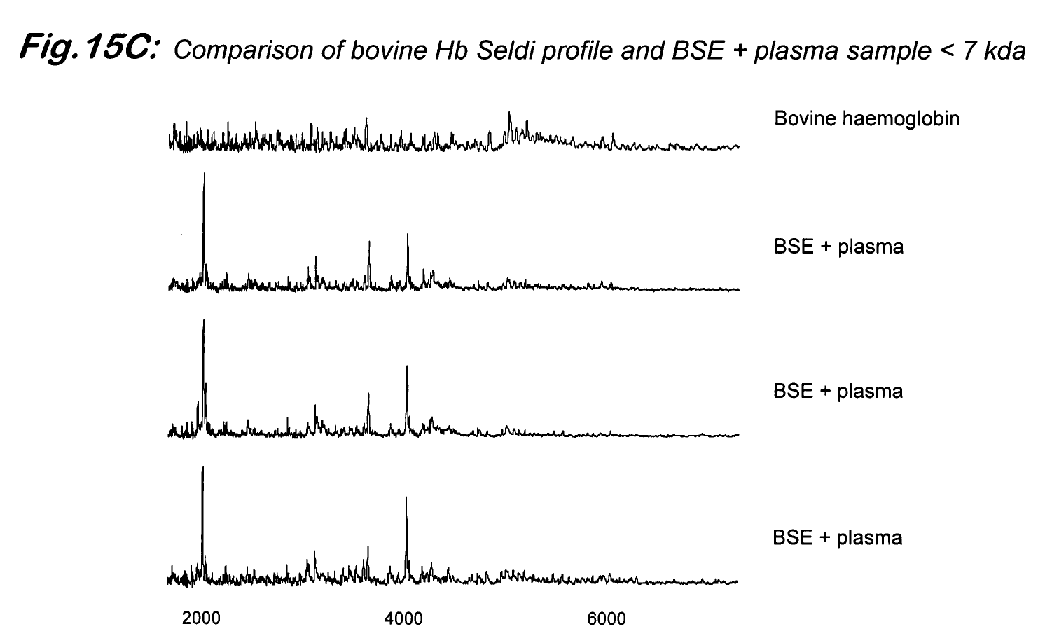

To further confirm the identity of the 15000 Da cluster as isoforms of bovine hemoglobin, bovine and human purified Hb were analyzed on Normal phase with SELDI. FIG. 14 shows the whole spectra obtained for both species, illustrating their similarity. FIGS. 15A and 15B point out within 4 representative spectra, how much the Hb profile looks like those of BSE+plasma samples on I MAC, whilst FIG. 15C highlights their difference under 7 kDa. These spectra led the inventors to conclude that 5 out of the 13 clusters highlighted in the earlier SELDI study of bovine plasma correspond to hemoglobin.

Each of the above cited publications is herein incorporated by reference to the extent to which it is relied on herein.

I/we claim:

1. A method of diagnosis of a transmissible spongiform encephalopathy (TSE) or the possibility thereof in a subject suspected of suffering from the TSE, which comprises subjecting a sample of body fluid taken from the subject to mass spectrometry, thereby to determine a test amount of a polypeptide in the sample, wherein the polypeptide is differentially contained in the body fluid of TSE-infected subjects and non-TSE-infected subjects, and has a molecular weight in the range of from 1000 to 100000; and determining whether the test amount is consistent with a diagnosis of TSE.

2. A method according to claim 1, in which the polypeptide is present in the body fluid of TSE-infected subjects and not present in the body fluid of non-TSE-infected subjects, whereby the presence of the polypeptide in a body fluid sample is indicative of TSE.

3. A method according to claim 1, in which the polypeptide is not present in the body fluid of TSE-infected subjects and present in the body fluid of non-TSE-infected subjects, whereby the non-presence of the polypeptide in a body fluid sample is indicative of TSE.

4. A method according to claim 1, in which the mass spectrometry is laser desorption/ionization mass spectrometry.

5. A method according to claim 4, in which the sample is adsorbed on a probe or on a protein chip array having an immobilized metal affinity capture (IMAC), hydrophobic, strong anionic or weak cationic exchange surface capable of binding the polypeptide.

6. A method according to claim 4, in which the polypeptide is determined by surface-enhanced laser desorption/ionization (SELDI) and time of flight mass spectrometry (TOF-MS).

7. A method according to claim 1, in which the body fluid is cerebrospinal fluid, plasma, serum, blood, tears, urine or saliva.

8. A method according to claim 1, in which a plurality of peptides is determined in the sample.

9. A method according to claim 1, in which the TSE is Creutzfeldt-Jakob disease (CJD).

10. A method according to claim 9, in which the TSE is sporadic Creutzfeldt-Jakob Disease (CJD) or variant Creutzfeldt-Jakob Disease (CJD).

11. A method according to claim 9, in which one or more polypeptides having a respective molecular weight of about 4780, about 6700, about 8600 or about 13375 is determined, and the presence of one or more of such polypeptides is indicative of CJD.

12. A method according to claim 9 in which one or more polypeptides having a respective molecular weight of about 3970, about 3990, about 4294, about 4478, about 10075, about 11730, about 14043 or about 17839 is determined, and the absence of one or more of such polypeptides is indicative of CJD.

13. A method according to claim 9, in which a polypeptide having a molecular weight of about 7770 is determined, and the presence of such polypeptide is indicative of CJD.

14. A method according to claim 9, in which a polypeptide having a molecular weight of about 3295, about 4315, about 4436, about 6200, about 8936, about 9107, about 9145, about 9185, about 9454 or about 13550 Da is determined, and the absence or decreased amount of one or more of such polypeptides is indicative of CJD.

15. A method according to claim 9, in which a polypeptide having a molecular weight of about 7574, about 7930, about 7975 or about 8020 Da is determined, and the presence or increased amount of one or more of such polypeptides is indicative of CID.

16. A method according to claim 1, in which the TSE is Bovine Spongiform Encephalopathy (BSE).

17. A method according to claim 16, in which the polypeptide has a molecular weight of about 10220, and the presence of the polypeptide is indicative of BSE.

18. A method according to claim 16, in which one or more polypeptides having a respective molecular weight of about 1010, 1100, 1125, 1365, 3645, 4030, 3890, 5820, 7520, 7630, 7980, 9950, 10250, 11600, 11800, 15000, 15200, 15400, 15600, 15900, 30000, 31000 and 31800 Da is determined, and the differential expression of one or more of such polypeptides is indicative of BSE.

19. A method according to claim 1, in which the TSE is scrapie.

20. A method of diagnosis, prognosis or therapy which comprises use of a polypeptide which is differentially contained in a body fluid of TSE-infected subjects and non-infected subjects, the polypeptide having a molecular weight in the range of from 1000 to 100000 and being determinable by mass spectrometry.

21. A method of diagnosis, prognosis or therapy which comprises use of a material which recognizes, binds to or has affinity for a polypeptide which is differentially contained in a body fluid of TSE-infected subjects and non-infected subjects, the polypeptide having a molecular weight in the range of trom 1000 to 100000 and being determinable by mass spectrometry.

22. A method according to claim 21, in which the material is an antibody or antibody chip.

23. An assay device for use in the diagnosis of TSE which comprises a plate having a location containing a material which recognizes, binds to or has affinity for a polypeptide which is differentially contained in a body fluid of TSE-infected subjects and non-infected subjects, the polypeptide having a molecular weight in the range of from 1000 to 100000 and being determinable by mass spectrometry.

24. An assay device for use in the diagnosis of TSE, which comprises a plate having a location containing an antibody that is specific for Cystatin C.

25. An assay device for use in the diagnosis of variant CJD, which comprises a plate having a location containing an antibody that is specific for Cystatin C and useful in the diagnosis of variant CJD.

26. An assay device for use in the diagnosis of sporadic CJD, which comprises a plate having a location containing an antibody that is specific for Cystatin C and useful in the diagnosis of sporadic CJD.

27. An assay device for use in the diagnosis of BSE, which comprises a plate having a location containing an antibody that is specific for a hemoglobin, a hemoglobin chain or a truncated chain or a fragment thereof having an immunological reaction to antibodies specific for bovine hemoglobin and useful in the diagnosis of BSE.

28. An assay device for use in the diagnosis of a TSE comprising a solid substrate having attached thereto an antibody that is specific for any of the following:
(i) a polypeptide that is differentially contained in the body fluid of TSE-infected subjects and non-TSE-infected subjects, and has a molecular weight in the range of from 1000 to 100000;
(ii) a polypeptide that is differentially contained in the body fluid of TSE-infected subjects and non-TSE-infected subjects, and is selected from those having a respective molecular weight of about 1010, 1100, 1125, 1365, 3645, 4030, 3890, 5820, 7520, 7630, 7980, 9950, 10250, 11600, 11800, 15000, 15200, 15400, 15600, 15900, 30000, 31000 and 31800 Da (iii) cystatin C;
(iv) a hemoglobin, a hemoglobin chain or a truncated chain or a fragment thereof which exhibits an immunological reaction to an antibody to bovine hemoglobin and is differentially contained in the body tissue of bovine TSE-infected subjects and non-bovine non-TSE-infected subjects.

29. A kit for use in diagnosis of TSE, comprising a probe for receiving a sample of body fluid, and for placement in a mass spectrometer, thereby to determine a test amount of a polypeptide in the sample, wherein the polypeptide is differentially contained in the body fluid of TSE-infected subjects and non-TSE-infected subjects, and has a molecular weight in the range of from 1000 to 100000.

30. A kit according to claim 29, in which the probe contains an adsorbent for adsorption of the polypeptide.

31. A kit according to claim 29, further comprising a washing solution for removal of unbound or weakly bound materials from the probe.

32. A method of diagnosis of a transmissible spongiform encephalopathy (TSE) or the possibility thereof in a subject suspected of suffering from the TSE, which comprises determining a test amount of a polypeptide in a sample of body fluid taken from the subject, wherein the polypeptide is differentially contained in the body fluid of TSE-infected subjects and non-TSE-infected subjects, and is Cystatin C; and determining whether the test amount is consistent with a diagnosis of TSE.

33. A method of diagnosis of a transmissible spongiform encephalopathy (TSE) or the possibility thereof in a subject suspected of suffering from the TSE, which comprises subjecting a sample of body fluid taken from the subject to mass spectrometry, thereby to determine a test amount of a polypeptide in the sample, wherein the polypeptide is differentially contained in the body fluid of TSE-infected subjects and non-TSE-infected subjects, and is Cystatin C; and determining whether the test amount is consistent with a diagnosis of TSE.

34. The method of claim 33, wherein the body fluid is CSF.

35. A method of diagnosis of a transmissible spongiform encephalopathy (TSE) or the possibility thereof in a bovine subject suspected of suffering from the TSE, which comprises determining a test amount of a polypeptide in a sample of body fluid taken from the subject, wherein the polypeptide is differentially contained in the body fluid of TSE-infected bovine subjects and non-TSE-infected subjects, and is a hemoglobin, a hemoglobin chain or a truncated chain or a fragment thereof which exhibits an immunological reaction to an antibody to bovine hemoglobin; and determining whether the test amount is consistent with a diagnosis of TSE.

36. A method of diagnosis of a transmissible spongiform encephalopathy (TSE) or the possibility thereof in a bovine subject suspected of suffering from the TSE, which comprises subjecting a sample of body fluid taken from the subject to mass spectrometry, thereby to determine a test amount of a polypeptide in the sample, wherein the polypeptide is differentially contained in the body fluid of TSE-infected bovine subjects and non-TSE-infected subjects, and is a hemoglobin, a hemoglobin chain or a truncated chain or a fragment thereof which exhibits an immunological reaction to an antibody to bovine hemoglobin; and determining whether the test amount is consistent with a diagnosis of TSE.

37. A method of providing an indication of a transmissible spongiform encephalopathy (TSE) or the possibility or progress thereof in a subject liable to suffer from the TSE, which comprises use as a marker of a level of at least one polypeptide that has a molecular weight in the range of from 1000 to 100000, is measurable or detectable in the body tissue by mass spectrometry and is differentially contained in the body fluid of TSE-infected subjects and non-TSE-infected subjects.

38. The method of claim 37, wherein said at least one polypeptide is selected from those having a respective molecular weight of about 1010, 1100, 1125, 1365, 3645, 4030, 3890, 5820, 7520, 7630, 7980, 9950, 10250, 11600, 11800, 15000, 15200, 15400, 15600, 15900, 30000, 31000 and 31800 Da.

39. The method of claim 37, in which the body fluid is cerebrospinal fluid, plasma, serum, blood, tears, urine or saliva.

40. A method of providing an indication of a transmissible spongiform encephalopathy (TSE) or the possibility or progress thereof in a subject liable to suffer from the TSE, which comprises use as a marker of a level of cystatin C measurable or detectable in a sample of body tissue by mass spectroscopy and differentially contained in the body tissue offSE-infected subjects and non-TSE-infected subjects.

41. The method of claim 40, wherein the body tissue is from a human subject.

42. The method of claim 40, wherein the body tissue is cerebrospinal fluid.

43. A method of providing an indication of a transmissible spongiform encephalopathy (TSE) or the possibility or progress thereof in a bovine subject liable to suffer from the TSE, which comprises use as a marker of a level of a hemoglobin, a hemoglobin chain or a truncated chain or a fragment thereof which exhibits an immunological reaction to an antibody to bovine hemoglobin, said level being measurable or detectable in a sample of body tissue by mass spectroscopy, and said hemoglobin, hemoglobin chain or truncated chain or fragment thereof being differentially contained in the body tissue of bovine TSE-infected subjects and non-bovine non-TSE-infected subjects.

44. The method of claim 43, wherein said hemoglobin, hemoglobin chain or truncated chain or fragment thereof has a molecular weight determinable by mass spectroscopy of about 15000 Da, 7500 Da or 3000 Da.

45. The method of claim 43, wherein the sample of body tissue is plasma.

46. The method of claim 43, wherein the sample of body tissue is from a living animal.

47. A bovine animal, or herd of said animals, that has or have been subjected to a test as defined in claim 43 and found to be free of a transmissible spongiform encephalopathy (TSE).
